(19) United States
(12) Patent Application Publication
(10) Pub. No.: US 2009/0053740 A1
(43) Pub. Date: Feb. 26, 2009

(54) AGGLUTINATION-BASED METHOD FOR FAST DETECTION, ISOLATION AND QUANTIFICATION OF APOPTOTIC CELLS

(75) Inventors: Rostyslav Stoika, Lviv (UA); Rostyslav Bilyy, Lviv (UA); Volodymyr Antonyuk, Lviv (UA)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/088,973

(22) PCT Filed: Oct. 31, 2006
(86) PCT No.: PCT/US06/42582

§ 371 Date: Aug. 28, 2008

Related U.S. Application Data
(60) Provisional application No. 60/731,768, filed on Oct. 31, 2005.

(51) Int. Cl. G01N 33/53; C12N 5/06

(52) U.S. Cl. 435/7.9; 435/7.1; 435/378

(57) ABSTRACT

The present invention relates to methods and kits for the detection, isolation and quantification of apoptotic cells based on the apoptotic cells' increased expression of alpha-D-mannose and/or beta-D-galactose containing glycoproteins. Lectins that bind to alpha-D-mannose and beta-D-galactose-rich glycoconjugates are used in the methods and kits for agglutination tests for the detection, isolation and quantification of apoptotic cells. Lectins may be used to stimulate the agglutination of cells and apoptosis may be detected by assessing the concentration of lectins required to agglutinate a cell population and comparing the concentration to predetermined values for intact cells and cells in various stages after induction of apoptosis.

### FIELD OF INVENTION

This invention relates to the detection, isolation and quantification of apoptotic cells based on utilization of increased expression of α-D-mannose and/or β-D-galactose containing glycoproteins in the apoptotic cells.

### BACKGROUND

All publications herein are incorporated by reference to the same extent as if each individual publication or patent application was specifically and individually indicated-to be incorporated by reference. The following description includes information that may be useful in understanding the present invention. It is not an admission that any of the information provided herein is prior art or relevant to the presently claimed invention, or that any publication specifically or implicitly referenced is prior art.

Apoptosis is a physiological process of programmed cell death intended to maintain appropriate quantities of cells within the living organism. Apoptosis is characterized by a sequence of distinct events ultimately leading to cell death and is the major process responsible for the breakdown of existing cells. In this way apoptosis plays a crucial role in the renewal of aged cells and removal of “sick” or virus-infected cells. Disturbances in this process may lead to different pathological states such as autoimmune disorders and cancer.

In the recent decade, a set of characteristic features attributable to apoptosis were discovered and used for the development of practical approaches for detection of apoptosis. Most of these features belong to measuring biochemical markers of apoptosis, located in nucleus, cytoplasm or mitochondria of the cell. However, such measurements inside the cell are time and resource consuming procedures.

Different cytomorphological and biochemical markers of apoptosis have been described (Trauth B C, Keesey J. Cell death. Guide to cell proliferation and apoptosis methods. Mannheim: Boehringer Mannheim; 1995. p 34-62; Molecular Biology of the Cell, 4th ed, Alberts B.; Johnson A.; Lewis J.; Raff M.; Roberts K.; Walter P., 2002, New York, Garland Science, 1536p). Various cytomorphological and biochemical markers of apoptosis are found in different compartments (plasma membrane, cytoplasm, nucleus, and mitochondria) of target cells. The most characteristic cytomorphological changes detected during apoptosis by means of light microscopy are cytoplasm condensation and chromatin aggregation, plasma membrane “bubbling” and formation of apoptotic bodies covered by an intact plasma membrane, and fragmentation of the nucleus. The most typical biochemical markers of apoptosis are: the expression of specific caspases and the appearance of cytochrome c in cytoplasm (Chang H Y, Yang X. Proteases for cell suicide: functions and regulation of caspases. Microbiol Mol Biol Rev 2000;64:821-846; Coher G M. Caspases: the executioners of apoptosis. Biochem J 1997; 326:1-16; Fujimura M, Morita-Fujimura Y, et al. Cytosolic redistribution of cytochrome c after focal cerebral ischemia in rats. J. Cereb Blood Flow Metab 1998;18:1239-1247; Perez-Pinzon M A, Xu G P, Born J, et al. Cytochrome C is released from mitochondria into the cytosol after cerebral anoxia or ischemia. J Cereb Blood Flow Metab 1999;19:39-43), the expressions of pro- and anti-apoptotic proteins of the Bcl-2 family in mitochondria (Reed J C. Bcl-2 and the regulation of programmed cell death. J Cell Biol 1994;124:1), and DNA fragmentation in the nucleus (Wyllie A. Glucocorticoid-induce thymocyte apoptosis is associated with endogenous endonuclease activation. Nature 1980;284:555-556). The plasma membrane of the apoptotic cells is believed to remain relatively intact.

In contrast to cytoplasm, nucleus and mitochondria, where numerous changes can be observed after apoptosis induction (Molecular Biology of the Cell, 4th ed, Alberts B.; Johnson A.; Lewis J.; Raff M.; Roberts K.; Walter P., 2002, New York, Garland Science, 1536p), only few markers of apoptosis were found in plasma membrane. The only well-documented changes in the membrane are the translocation of phosphatidylserine to the external side of the plasma membrane, which can be detected by Annexin V-specific binding (Fadok V A, Voelker D R, Campbbell P A, et al. Exposure of phosphatidylserine on the surface of apoptotic lymphocytes triggers specific recognition and removal by macrophages. J Immunol 1992;148:2207-2216; Zhang G, Gurtu V, Kain S R, Yan G. Early detection of apoptosis using a fluorescent conjugate of annexin V. Biotechniques 1997;23:525-531), and the expression of Fas and tumor necrosis factor membrane receptors in the apoptotic cells (Fadok V A, Voelker D R, Campbbell P A, et al. Exposure of phosphatidylserine on the surface of apoptotic lymphocytes triggers specific recognition and removal by macrophages. J Immunol 1992;148:2207-2216; Orlinick J R, Chao M V. TNF-related ligands and their receptors. Cell Signal 1998;10:543-551). An ability of protein annexin V to specifically bind phosphatidylserine was used for the development of new methods of apoptosis detection (U.S. Pat. No. 5,834,196).

Although the plasma membrane is an easily accessible cellular compartment, relatively little is known about its changes during apoptosis. Glycoproteins of plasma membrane constitute a very sophisticated system and the inventors found that the expression of some plasma membrane glycoproteins is changed during apoptosis.

It has been reported that short-time acid pretreatment of apoptotic cells and their subsequent staining with FITC-labeled lectin from Narcissus pseudonarcissus can be a reliable tool for early detection of apoptosis (Heyder P, Gaipl U S, Beyer T D, Voll R E, Kern P M, Stach C, Kalden J R, Herrmann M, 2003. Early detection of apoptosis by staining of acid-treated apoptotic cells with FITC-labeled lectin from Narcissus pseudonarcissus. Cytometry 55A:86-93). Treatment of human apoptotic lymphocytes with FITC-labeled ConA revealed an increase in sugar residues on plasma membrane of these cells (Chionna A, Dwikat M, Panzarini E, Tenuzzo B, Carla E C, Verri T, Pagliara P, Abbro L, Dini L, 2003. Cell shape and plasma membrane alterations after static magnetic fields exposure. Eur J Histochem 47:299-308).

Lectins are carbohydrate-binding proteins that possess different carbohydrate specificities (Lutsik A D, Detjuk E S, Lutsik M D. Lectins in histochemistry [in Russian]. Lvov: Lvov University Press; 1989). They are widely used in histology and cytology for different purposes, such as the identification of carbohydrate moieties of membrane components (Kawiak J, Skorski T, Ciechanowicz A, et al. Cytochemical characterization of mouse L1210 leukemia. Immunol Invest 1988;17:543-550), tumor cell destruction (Khopade A J, Nandakumar K S, Jain N K. Lectin-functionalized multiple emulsions for improved cancer therapy. J Drug Target 1998;6:285-292), and the induction of cellular growth and differentiation (Lucas T, Krugluger W, Samorapoompichit P, et al. Self-renewal, maturation, and differentiation of the rat myelomonocytic hematopoietic stem cell. FASEB J 1999; 13:263-272).

Presently, apoptosis detection methods such as that which is described in U.S. Pat. No. 5,834,196, BioCat Lectin-Narcissus-Pseudonarcissus Apoptotic Necrosis-Detection Kit (Heidelberg, Germany; covered by German Patent DE 10053521B4), and others may be reliable for apoptosis detection. However, there are disadvantages such as high cost of analysis and the necessity of using complicated and bulky devices for the detection, which requires specially equipped laboratories for such testing. For example the BioCat Detection kit requires acid treatment of cells prior to the detection of apoptotic cells and flow cytometry to detect the presence of apoptotic cells. Thus, there is a need for improvement in the art for compositions, methods and kits for the detection, quantification and isolation of apoptotic cells.

### SUMMARY OF THE INVENTION

The following embodiments and aspects thereof are described and illustrated in conjunction with methods and kits are meant to be exemplary and illustrative, not limiting in scope.

The current invention includes methods and kits for the detection, quantification and isolation of apoptotic cells utilizing agglutination properties. The inventors have found that the concentration of lectins required for agglutination is inversely proportional to the amount of α-D-mannose and β-D-galactose-rich glycoconjugates in the cell membrane.

Various embodiments of the present invention provide for methods for detection apoptotic cells in a sample of cells. In one embodiment, the method for detecting apoptotic cells in a sample of cells comprises providing a lectin that possesses at least two carbohydrate-recognition domains; and adding a quantity of the lectin to the sample of cells, wherein the observation of agglutinating cells in the sample indicates the presence of apoptotic cells cells in the sample indicates the presence of apoptotic cells.

In one embodiment, the quantity of the lectin may be less than a quantity of lectin that is capable of causing agglutination of intact cells.

In one embodiment, the lectin may be labeled with a label selected from the group consisting of enzymatic label, biotin, fluorescent and combinations thereof, and the method may further comprise detecting the presence of the label, wherein the presence of the label indicates the presence of apoptotic cells.

In another embodiment, the method may further comprise determining a minimum quantity of lectin that causes agglutination of the cells; and comparing the minimum quantity of lectin to predetermined quantities of lectin that cause agglutination of intact cells and apoptotic cells, wherein if the minimum quantity of lectin is less than the predetermined quantity of lectin that causes agglutination of intact cells, the presence of apoptotic cells is indicated.

In one embodiment, the lectin may be capable of simultaneously binding at least two cells. In another embodiment, the lectin may be capable of binding to an α-D-mannose-rich glycoprotein, a β-D-galactoste-rich glycoprotein, or both. In various embodiments, the lectin may be selected from the group consisting of lectins from Pisum sativum (PSL), Polygonatum multiforum (PMRL), Galanthus nivalis (GNA), Ricinus communis (RCA-120), Viscum album (VAA), and combinations thereof. In one embodiment, the lectin may be from Viscum album.

In one embodiment, detecting apoptotic cells may comprise detecting apoptotic cells after about 12 hours after induction of apoptosis.

In another embodiment, the lectin may be from Pisum sativum (PSL) and the predetermined quantity for intact cells may be about eight times higher than the predetermined quantity for apoptotic cells. In another embodiment, the lectin may be from Polygonatum multiforum (PMRL) and the predetermined quantity for intact cells may be from about four to about eight times higher than the predetermine quantity for apoptotic cells. In another embodiment, the lectin may be from Viscum album (VAA) and the predetermined quantity for intact cells may be from about 4 times to about 128 times higher than the predetermined quantity for apoptotic cells.

In one embodiment, the sample of cells may comprise human lymphocytes.

Other methods of the present invention provide for quantifying the amount of apoptotic cells in a sample of cells. In one embodiment, the method of quantifying the amount of apoptotic cells in a sample of cells comprises providing a lectin that possesses at least two carbohydrate-recognition domains; determining a minimum quantity of the lectin that is capable of causing the sample of cells to agglutinate; and comparing the minimum quantity of lectin to predetermined quantities of lectin that cause agglutination of intact cells and apoptotic cells in various stages after induction of apoptosis to determine the quantity of apoptotic cells in the sample of cells.

In one embodiment, the lectin may be labeled with a label selected from the group consisting of enzymatic label, biotin label, fluorescent label and combinations thereof, and the method further comprise detecting the presence of the label, wherein the presence of the label indicates the presence of apoptotic cells.

In one embodiment, the lectin may be capable of simultaneously binding at least two cells.

In another embodiment, the lectin may be capable of binding to an α-D-mannose-rich glycoprotein, a β-D-galactoste-rich glycoprotein, or both.

In another embodiment, the lectin may be selected from the group consisting of lectins from Pisum sativum (PSL), Polygonatum multiforum (PMRL), Galanthus nivalis (GNA), Ricinus communis (RCA-120), Viscum album (VAA), and combinations thereof. In one embodiment the lectin may be from Viscum album (VAA).

In one embodiment, quantifying the amount of apoptotic cells may comprise quantifying the amount of apoptotic cells after about 12 hours after induction of apoptosis.

In one embodiment, predetermined quantities of lectin that cause agglutination of intact cells and apoptotic cells in various stages after induction of apoptosis may be determined by correlating quantities of lectin that cause agglutination of control samples of cells within known amounts of apoptotic cells.

Other embodiments of the present invention provide for methods for isolating apoptotic cells from a sample of cells. In one embodiment, the method for isolating apoptotic cells from a sample of cells comprises providing a conjugated lectin; contacting the sample of cells to the conjugated lectin to generate a fraction of cells that are bound to the conjugated lectin and a fraction of cells that are not bound to the conjugated lectin; and separating the fraction of cells that are bound to the conjugated lectin from the conjugate to produce a fraction of cells comprising the apoptotic cells.

In one embodiment, the conjugated lectin may be a lectin-conjugated support medium. In other embodiments, the conjugate may be a label selected from the group consisting of enzymatic label, biotin, fluorescent and combinations thereof, and the method may further comprise detecting the presence of the label, wherein the presence of the label indicates the presence of apoptotic cells.

In one embodiment, the lectin may be capable of simultaneously binding at least two different cells. In another embodiment, the lectin may be capable of binding to an α-D-mannose-rich glycoprotein, a β-D-galactose-rich glycoprotein, or both. In other embodiments, the lectin may be selected from the group consisting of lectins from Pisum sativum (PSL), Polygonatum multiforum (PMRL), Galanthus nivalis (GNA), Ricinus communis (RCA-120), Viscum album (VAA), and combinations thereof.

Still further embodiments of the present invention provide for kits for the detection and/or quantification of apoptotic cells in a sample of cells. The kits may comprise a quantity of a lectin that possesses at least two carbohydrate-recognition domains; and instructions to use the quantity of lectin to detect and/or quantify apoptotic cells.

In one embodiment, the lectin may be capable of simultaneously binding at least two cells. In another embodiment, the lectin may be capable of binding to an α-D-mannose-rich glycoprotein, a β-D-galactose-rich glycoprotein, or both. In other embodiments, the lectin may be selected from the group consisting of lectins from Pisum sativum (PSL), Polygonatum multiforum (PMRL), Galanthus nivalis (GNA), Ricinus communis (RCA-120), Viscum album (VM), and combinations thereof. In one embodiment, the lectin may be from Pisum sativum (PSL) or Viscum album (VAA).

In another embodiment, the instructions to use the quantity of lectin to detect apoptotic cells may comprise instructions to add a quantity of the lectin to the sample of cells; and detect the presence of agglutination of cells in the sample, wherein the quantity of lectin is less than a quantity of lectin that is capable of causing agglutination of intact cells and the presence of agglutination of cells indicates the presence of apoptotic cells.

In another embodiment, the instructions may further comprise instructions to: determine a minimum quantity of lectin that causes agglutination of the cells; and compare the minimum quantity of lectin to predetermined quantities of lectin that cause agglutination of intact cells and apoptotic cells, wherein the minimum quantity of lectin that is less than the predetermined quantity of lectin that causes agglutination of intact cells indicates the presence of apoptotic cells.

In one embodiment, the lectin may be from Pisum sativum (PSL) and the predetermined quantity for intact cells is about eight times higher than the predetermined quantity for apoptotic cells. In another embodiment, the lectin may be from Polygonatum multiforum (PMRL) and the predetermined quantity for intact cells is from about four to about eight times higher than the predetermine quantity for apoptotic cells. In another embodiment, the lectin may be from Viscum album (VAA) and the predetermined quantity for intact cells is from about 4 times about 128 times higher than the predetermined quantity for apoptotic cells.

In another embodiment, the instructions to use the quantity of lectin to quantify apoptotic cells may comprise instructions to determine a minimum quantity of the lectin that is capable of causing the sample of cells to agglutinate; and compare the minimum quantity of lectin to a predetermined quantities of lectin that cause agglutination of intact cells and apoptotic cells in various stages after induction of apoptosis to determine the quantity of apoptotic cells.

In another embodiment, the predetermined quantities of lectin that cause agglutination of intact cells and apoptotic cells in various stages after induction of apoptosis may be determined by correlating quantities of lectin that cause agglutination of control samples of cells within known amounts of apoptotic cells.

Additional embodiments of the present invention provide for kits for isolating apoptotic cells from a sample of cells. The kits may comprise a quantity of conjugated lectins; and instructions to use the quantity of conjugated lectins to isolate apoptotic cells. In one embodiment, the conjugated lectin may be a lectin-conjugated support medium.

In one embodiment, the instructions may comprise instructions to contact the sample of cells to the conjugated lectin to generate a fraction of cells that are bound to the conjugated lectin and a fraction of cells that are not bound to the conjugated lectin; separate the fraction of cells that are bound to the conjugated lectin and the fraction of cells that are not bound to the conjugated lectin; and separate the fraction of cells that are bound to the conjugated lectin from the conjugated lectin.

In one embodiment, the conjugate may be a label selected from the group consisting of enzymatic label, biotin, fluorescent and combinations thereof, and the instructions may further comprise instructions to detect the presence of the label, wherein the presence of the label indicates the presence of apoptotic cells. in one embodiment, the lectin may be capable of simultaneously binding at least two cells. In another embodiment, the lectin may be capable of binding to an α-D-mannose-rich glycoprotein, a β-D-galactose-rich glycoprotein, or both. In another embodiment, the lectin may be selected from the group consisting of lectins from Pisum sativum (PSL), Polygonatum multiforum (PMRL), Galanthus nivalis (GNA), Ricinus communis (RCA-120), Viscum album (VAA), and combinations thereof.

Other features and advantages of the invention will become apparent from the following detailed description, taken in conjunction with the accompanying figures, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts densitometry (mean±standard error) of normal (open columns) and apoptotic (solid columns) murine leukemia L1210S cells (apoptosis was induced by 100 μg/ml of methotrexate) in accordance with an embodiment of the present invention. Cells were stained with different horseradish peroxidase-labeled lectins. I: sugar inhibitor (35 mM; aMMan for PSL and lactose for RCA-120). *P<0.05. **P<0.01. ***P<0.001.

FIG. 2 depicts lectin cytochemical analysis of L1210 cells in accordance with an embodiment of the present invention. A: Intact cells stained with RCA-120. B: Apoptotic cells stained with RCA-120. C: Apoptotic cells stained with RCA-120 in the presence of lactose. D: Intact cells stained with PSL. E: Apoptotic cells stained with PSL. F: Differential staining of apoptotic bodies by PSL lectin. A to E were contrasted with NiCl₂.

FIG. 3 depicts densitometry (mean i standard error) of normal (open columns) and apoptotic (solid columns) murine leukemia L1210S cells (apoptosis was induced by methotrexate) in accordance with an embodiment of the present invention. Hatched columns represent apoptosis induced by cisplatin. Cells were stained with different horseradish peroxidase-labeled lectins. A: L1210S cells with apoptosis induced by 0.5 μg/ml of cisplatin. L1210R cells with apoptosis induced by 100 μg/ml of methotrexate (B), 0.5 μg/ml of cisplatin, (C), and 5 μg/ml of cisplatin (D). *P<0.05. **P<0.01. ***P<0.001.

FIG. 4 depicts sodium dodecyl sulfate polyacrylamide gel electrophoresis and lectin blotting with horseradish peroxidase-labeled PSL of soluble (lane 1) and membrane (lane 2) fractions of L1210S cells in accordance with an embodiment of the present invention.

FIG. 5 depicts DNA gel electrophoresis of murine leukemia L1210S (lanes 1 and 2) and L1210R (lanes 3-5) cells in accordance with an embodiment of the present invention. Lanes 1 and 3: untreated cells; lanes 2 and 4: cells treated with 0.5 μg/ml of cisplatin; lane 5: cells treated with 5 μg/ml of cisplatin.

FIG. 6 depicts densitometry (M±m) of murine fibroblasts of L929 line under action of different inducers of apoptosis and using different methods of cell detachment in accordance with an embodiment of the present invention. Cells were stained with different horseradish peroxidase-labeled lectins. (I)-sugar inhibitor (35 mM) (αMMan. for PSL and lactose for RCA).

FIG. 7 depicts glycoprotein expression in normal and apoptotic cells of MCF-7 (wild type, wt; and doxorubicine-resistant, DOXIR) (A-C) and Jurkat (D-F) cell lines in accordance with an embodiment of the present invention. (A, D) Densitometry of cells, stained with different HRP-labeled lectin, demonstrates increased binding of mannose and galactose-specific lectins by apoptotic cells. (B, E) Intact cells. (C, F) Apoptotic cells are characterized by more intense staining. B and C stained with HRP-WGA, E and F stained with HRP-PSL. (I) sugar inhibitor (35 mM) of lectin—αMMan for PSL.

FIG. 8 depicts DNA gel electrophoresis of Jurkat cells in accordance with an embodiment of the present invention. (1) untreated cells; (2) treated with dexamethasone (1 μM, 24 h); (3) treated with cisplatin (5 μg/ml, 24 h).

FIG. 9 depicts dose and time dependence of glycoprotein expression during apoptosis in accordance with an embodiment of the present invention. (A) Effect of different concentrations of cisplatin on quantity of live L1210 cells. (B) Effect of different concentrations of cisplatin on glycoprotein expression in L1210 cells. (C) Timedependence of glycoprotein expression in apoptotic L929 cells. Cells were stained with HRP-labeled PSL, RCA, VAA and WGA lectins.

FIG. 10 depicts the effect of 2 h pretreatment with RCA and VAA lectins on L1210 cells' staining with HRP-labeled RCA and VAA lectins in accordance with an embodiment of the present invention.

FIG. 11 depicts agglutination of non-apoptotic and apoptotic L1210 cells by PMRL lectin in accordance with an embodiment of the present invention.

FIG. 12 depicts isolation of apoptotic L1210 cells in accordance with an embodiment of the present invention. (A) Scheme of isolation of intact and apoptotic cells from their mixed populations. (B) Fluorescent microscopy of L1210 cells after isolation procedure, using PSL-conjugated agarose, negative fraction (cells not bound to PSL-agarose) represents intact cells. Positive fraction (cell bound to PSL-agarose under described incubation conditions) represents “apoptotic” cells.

FIG. 13 depicts agglutination of intact (I) and apoptotic (A) L1210 cells by PSL, VM and PMRL lectins in accordance with an embodiment of the present invention. Apoptosis was induced by cisplatin (5 μg/ml, 24 h).

FIG. 14 depicts agglutination of intact (I) and apoptotic (A) Jurkat cells by PSL, VM, RCA and PMRL lectins in accordance with an embodiment of the present invention. Apoptosis was induced by etoposide, 1 μM, 24 h.

FIG. 15 depicts the use of VAA lectin-stimulated agglutination for the detection of apoptosis in lymphocyte suspensions isolated from peripheral blood of “healthy” donors and patients with autoimmune diseases in accordance with an embodiment of the present invention. D: healthy donor, <1% of apoptotic cells; 1: Patient N. G., 1.06% of apoptotic cells; 2: Patient T. O., 6.7% of apoptotic cells.

FIG. 16 depicts the use of VAA lectin-stimulated agglutination for the detection of apoptosis in lymphocyte suspensions isolated from peripheral blood of “healthy” donor (D) and patient V. P. 1 with active articular form of polyarthritis, before (A) and after a 14-day course of chemotherapy (B) in accordance with an embodiment of the present invention.

FIG. 17 depicts the quantification of number of live and apoptotic cells. Apoptosis in L1210 cells was induced by cisplatin used in different concentrations, namely 0.05, 0.5 and 5 mg/ml. A percentage of live cells in their population after apoptosis induction was calculated. Number of apoptotic cells equals “% apoptotic cells=100% cells−% alive cells”. Concentration of VAA lectin needed to agglutinate cells in their population was detected. A dependence of the percentage of live cells upon specific lectin concentration needed for the agglutination is shown. A sigmoidal fit of the dependence is proposed.

FIG. 18 depicts the use of FITC-labeled PSL lectin for the detection of apoptotic cells of human lung adenocarcinoma A549 line by means of fluorescent microscopy. Apoptosis was induced by different concentrations of cisplatin.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N Y 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3^{rd} ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

The current invention includes methods and kits for the detection, quantification and isolation of apoptotic cells utilizing agglutination properties.

Various embodiments include compositions comprising lectins. The lectins may be used to stimulate the agglutination of cells. In further embodiments lectins that possess at least two carbohydrate-recognition domains per molecule and thus are capable of simultaneously binding at least two cells may be used. In particular embodiments, lectins that can specifically bind different glycoconjugate residues are used. Examples of lectins that may be suitable for use in connection with various embodiments of the invention include, but are not limited to, Laburnum anagyroides bark agglutinin (LABA), Phaseolus vulgaris agglutinin (PHA-E), Pisum sativum lectin (PSL), Ricinus communis agglutinin (RCA-120 or RCA), Solanum tuberosum agglutinin (STA), Triticum vulgaris agglutinin (wheat germ agglutinin, WGA), Viscum album agglutinin (VAA), Canavalia ensiformis lectin (concanavalin A, ConA), Helix pomatia lectin (HPL), Galanthus nivalis agglutinin (GNA), Narcissus pseudonarcissus agglutinin (NPA), Polygonatum multiflorum rhizome lectin (PMRL), Leucojum verum agglutinin (LVA), Sambucus nigra agglutinin (SNA), Lens cilinaris aggutinin (LCA), Phytolacca americana agglutinin (PLA), and L-fucose specific lectin from river perch (Perca fluviatilis) hardroe. Particularly useful lectins may be PSL, PMRL, VAA, RCA, and GNA, which can bind α-D-mannose- and/or β-D-galactose-rich glycoconjugates. Equivalents, synthetic variants, chemical analogs and the like of any of the foregoing or combinations thereof may be used in connection with alternative embodiments of the present invention.

Additional embodiments include methods for detection and/or quantification of apoptotic cells. Apoptosis may be detected and/or quantified by adding a quantity of one or more lectins to a sample of cells, wherein an appreciable amount of agglutination of the cells indicates the presence of apoptotic cells in the sample of cells. As used herein, an “appreciable amount” of agglutination means an amount of agglutination wherein agglutinates are clearly seen at an about 4 to 5 fold magnification, and particularly at about 4.8 fold magnification. References herein to “agglutinating” shall have a similar meaning. Alternatively, apoptosis may be detected and/or quantified by assessing the concentration of lectins required to cause an appreciable amount of agglutination of a cell population and comparing the concentration to predetermined values for intact cells and/or cells in various stages after induction of apoptosis. In one embodiment, the predetermined quantities may be established by correlating quantities of lectin that cause agglutination of control samples of cells within known amounts of apoptotic cells. See, e.g., Example 32. The concentration of lectins required for agglutination is inversely proportional to the amount of α-D-mannose and β-D-galactose-rich glycoconjugates in the cell membrane. In a particular embodiment, the concentration of lectins required to cause an appreciable amount of agglutination of non-apoptotic cells may be higher than that needed to do so with apoptotic cells. For example: (1) PMRL and PSL lectin concentration needed to agglutinate non-apoptotic L1210 cells were about 8 times higher than that needed to agglutinate apoptotic L1210 cells when using the agglutination method utilizing slide glass and microscope examination (see, e.g., Example 25); (2) VAA lectin concentration needed to agglutinate non-apoptotic L1210 cells was about 128 times higher than that needed to agglutinate apoptotic L1210 cells when using the agglutination method utilizing 96-well immunological plates and transmissive scanner examination (see, e.g., Example 27); (3) PMRL, lectin concentration needed to agglutinate non-apoptotic L1210 cells was about 4 times higher than that needed to agglutinate apoptotic L1210 cells when using the agglutination method utilizing 96-well immunological plates and transmissive scanner examination (see, e.g., Example 27); (4) VAA lectin concentration needed to agglutinate non-apoptotic Jurkat cells was about 16 times higher than that needed to agglutinate apoptotic Jurkat cells when using the agglutination method utilizing 96-well immunological plates and transmissive scanner examination (see, e.g., Example 28); and (5) VAA lectin concentration needed to agglutinate non-apoptotic polysteoarthrtis lymphocytes was about 64 times higher than that needed to agglutinate healthy lymphocytes when using the agglutination method utilizing 96-well immunological plates and transmissive scanner examination (see, e.g., Example 29).

In another embodiment, enzymatically labeled lectins (e.g., peroxidase, phosphatase) may be used for detection of apoptosis by light microscopy in cell smears. In another embodiment, biotinylated lectins (e.g., avidin, streptavaidin) may be used for detection of apoptosis by light microscopy, fluorescent microscopy and/or flow cytometry. In another embodiment, fluorescent dye-labeled lectins (e.g., FITC, Texas red) may be used for detection of apoptotic cells in fixed smears and live cell suspension by using fluorescent microscopy. (See, e.g., Example 33). Enzymatic, fluorescent labeling or biotinilation may be performed according to standard procedures; for example, those described in Hermanson G. T. Bioconjugate Techniques, Academic Press, San Diego, Calif., USA, 1996; Rhodes J. M. and Milton J. D. Lectin methods and protocols, Humana Press, 1997.

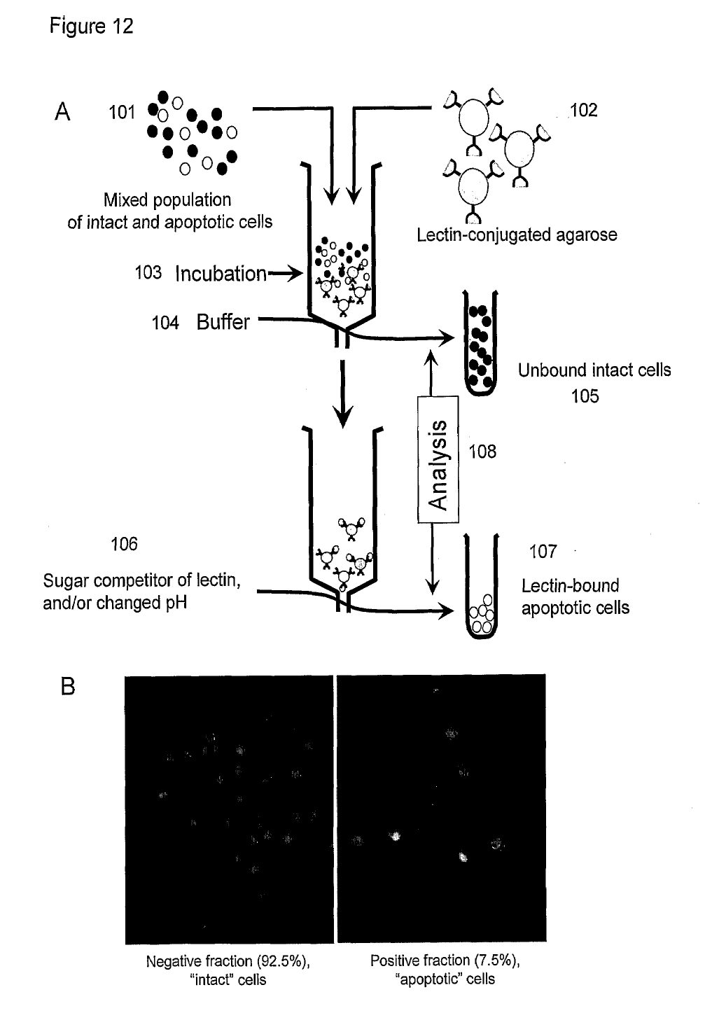

Other embodiments include isolation of apoptotic cells by the use of lectin-affinity methods. For example, a cell sample may be added to a lectin-conjugated coarse-grained agarose followed by an incubation period. The suspension may be transferred to a column with an inert sieve that allows the passing of unbound cells (e.g., non-apoptotic cells) but may retard the agarose particles in the bottom of the column. The column may then be washed with a buffer to release and collect the lectin-bound cells (e.g., apoptotic cells). (See, e.g., Example 18, FIG. 12A.) In another embodiment flow cytometric study or FACS of apoptotic cells may be used. Other standard cell sorting techniques may be modified or adapted with the lectin-affinity methods of the present invention, as will be readily appreciated by those of skill in the art and can be implemented by routine experimentation. In an alternative embodiment, fluorescently-labeled lectins may be used in cytometric study or FACS of apoptotic cells. Appropriate label may be selected according to the desired experimental conditions. The label may be attached to the lectins using standard procedures; for example, those described in Hermanson G. T. Bioconjugate Techniques, Academic Press, San Diego, Calif., USA, 1996; Rhodes J. M. and Milton J. D. Lectin methods and protocols, Humana Press, 1997.

In one embodiment, as depicted in FIG. 12A, a method of isolating apoptotic cells may comprise step 101 of providing a mixed population of intact and apoptotic cells; step 102 of providing lectin-conjugated agarose beads; step 103 of incubating the mixed population of cells and the lectin-conjugated beads; step 104 of eluting the unbound intact cells 105 with a buffer; step 106 of separating the apoptotic cells from the beads (e.g., adding a sugar competitor of lectin and/or changing the pH); and step 107 of eluting the lectin-bound apoptotic cells. Step 108 may be performed to analyze the unbound intact cells and/or the lectin-bound apoptotic cells.

In alternative embodiments, the lectin-affinity methods may comprise using materials other than agarose, for example, glass beads. One skilled in the art will recognize other appropriate materials that are suitable for use for the lectin-affinity methods, as noted above.

Still further embodiments include methods for the induction of apoptosis, which may be used in connection with the isolation, detection and quantification methods described herein. Induction of apoptosis may be accomplished by various methods; for example, by hyperthermia, radiation, use of methotrexate, use of cisplatin, and/or use of dexamethasone. Further methods will be readily ascertained by those of skill in the art. In a further embodiment, cell viability may be controlled by trypan-blue exclusion test. Further embodiments may include the use of sugar inhibitors, for example α-Methyl-D-mannopyranoside and/or 4-O-(a-D-galactopyranosyl)-D-glucopyranose.

The present invention is also directed to kits for the detection, isolation, and/or quantification of apoptotic cells. The kit is useful for practicing the inventive methods of detecting, isolating and/or quantifying apoptotic cells. The kit is an assemblage of materials or components, including at least one of the inventive compositions. Thus, in some embodiments, the kit contains a composition including one or more lectins, as described herein. In other embodiments, the kits contain lectin-conjugated beads as described herein.

The exact nature of the components configured in the inventive kit depends on its intended purpose. In one embodiment, the kit is configured particularly for the purpose of detecting and/or quantifying and/or isolating apoptotic cells.

Instructions for use may be included in the kit. “Instructions for use” typically include a tangible expression describing the technique to be employed in using the components of the kit to effect a desired outcome, such as to detect, isolate or quantify apoptotic cells. Instructions for use may include, but are not limited to, instructions to add a quantity of the lectin to the sample of cells; detect the presence of agglutination of cells in the sample, wherein the quantity of lectin is less than a quantity of lectin that is capable of causing agglutination of intact cells and the presence of agglutination of cells indicates the presence of apoptotic cells; determine a minimum quantity of lectin that causes agglutination of the cells; and compare the minimum quantity of lectin to predetermined quantities of lectin that cause agglutination of intact cells and apoptotic cells, wherein the minimum quantity of lectin that is less than the predetermined quantity of lectin that causes agglutination of intact cells indicates the presence of apoptotic cells. In alternate embodiments of the kit, the instructions to use may include, but are not limited to instructions to determine a minimum quantity of the lectin that is capable of causing the sample of cells to agglutinate; and to compare the minimum quantity of ectin to predetermined quantities of lectin that cause agglutination of intact cells and apoptotic cells in various stages after induction of apoptosis to determine the quantity of apoptotic cells. In other embodiments of the kit, the instructions to use may include, but are not limited to, instructions to contact the sample of cells to a lectin-conjugated support medium to generate a fraction of cells that are bound to the lectin-conjugated support medium and a fraction of cells that are not bound to the lectin-conjugated support medium; separate the fraction of cells that are bound to the lectin-conjugated support medium and the fraction of cells that are not bound to the lectin-conjugated support medium; and separate the fraction of cells that are bound to the lectin-conjugated support medium from the lectin-conjugated support medium, wherein the fraction of cells that are separated from the lectin conjugated support medium comprises apoptotic cells.

Optionally, the kit also contains other useful components, such as, lectin-conjugated support medium, culture medium, antibiotics, compositions to induce apoptosis, sugar inhibitors, acetone, buffers, slides, test tubes, Petri dishes, columns, staining compositions such as Acridine orange, multiple well plates such as a 96-well immunological plates, diluents, syringes, pipetting or measuring tools, siliconized tubes or other useful paraphernalia as will be readily recognized by those of skill in the art.

The materials or components assembled in the kit can be provided to the practitioner and stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in laboratory kits. As used herein, the term “package” refers to a suitable solid matrix or material such as glass, plastic, paper, foil, and the like, capable of holding the individual kit components. Thus, for example, a package can be a glass vial used to contain suitable quantities of a composition containing lectins. The packaging material generally has an external label which indicates the contents and/or purpose of the kit and/or its components.

The current invention is based on the increased expression of α-D-mannose-and β-D-galactose-containing glycoprotein(s) in apoptotic cells, and on the detection of apoptotic cells based on an agglutination test. In this test, the inventors use specific lectins that may possess at least 2 carbohydrate-recognition domains per molecule and thus are capable of simultaneously binding at least two different cells. These lectins can bind α-D-mannose- and/or β-D-galactose-rich glycoproteins with specificity. The developed agglutination test determines the minimum concentration of a specific lectin that agglutinates the apoptotic cells without significantly affecting the intact cells. The lectin concentration needed for agglutination is inversely proportional to the quantity of corresponding glycoproteins in the plasma membranes of either intact or apoptotic cells. Thus, the levels of α-D-mannose- and β-D-galactose-rich glycoproteins may be assessed on the basis of lectin concentration. The obtained values may be compared with predetermined values for intact cells and cells after induction of apoptosis (different levels of apoptosis induction), and the degree of apoptosis in a cell population may thus be estimated. The inventors showed the usefulness of α-D-mannose-specific lectins from Pisum sativum (PSL) and rhizome of Polygonatum multiflorum (PMRL), and β-D-galactose-specific lectins from Ricinus communis (120 kDa, RCA) and Viscum album (VAA) in the agglutination test. An embodiment of the present invention therefore enables fast and convenient detection of apoptotic cells.

Increased expression of α-D-mannose- and β-D-galactose-containing. glycoproteins in the plasma membrane of apoptotic cells have not been used for detection of such cells. The present invention is based on combining a conventional agglutination test with the above noted phenomenon for a fast and convenient method of apoptosis detection. This method does not require expensive reagents and equipment for detecting the apoptotic cells. Among the invention's advantages include, but are not limited to (1) early detection of apoptosis (e.g., starting 12 hours after its induction) and (2) low cost of analysis as compared to the closest analogue—Annexin V test.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

The inventors used specific plant lectins to follow the expression of plasma membrane glycoproteins at apoptosis. The inventors found that the levels of α-D-mannose-rich glycoconjugates specific to Pisum sativum lectin (PSL) and β-D-galactose-rich glycoconjugates, specific to Ricinus communis agglutinin (RCA-120; 120 kDa) were significantly increased in the plasma membrane of the apoptotic murine leukemia cells of L1210 line.

### Example 2

The inventors further characterized changes in glycoprotein expression during apoptosis to determine whether the results of lectinocytochemical detection of apoptotic L1210 cells can be generalized for apoptotic cells derived from other tissues and species. The inventors also studied how these changes depended on time of action and on the dose and nature of an apoptosis-inducing agent, as well as on the manner of cell detachment (trypsinization or mechanical rubbing). Relative levels of glycoprotein expression in normal and apoptotic cells were determined by lectinocytochemical analysis using 15 HRP-labeled lectins, and also by agglutination analysis. Isolation of apoptotic cells was carried out by PSL-linked agarose. The results obtained suggest that an increase in α-D-mannose- and β-D-galactose rich glycoprotein levels can be considered a universal marker of apoptotic cells, and thus, can be further used for isolation and identification of these cells.

The inventors show that an increase in levels of mentioned glycoproteins is a universal feature of apoptotic cells, independent of cell or tissue origin or manner of apoptosis induction. This feature of apoptotic cells was demonstrated as early as 12 hours after the induction of apoptosis.

The present data suggest that an increase in the expression of membrane glycoproteins containing α-D-mannose, β-D-galactose, or N-acetyl-glucosamine, and a simultaneous decrease in sialic acid positively correlates with the incidence of apoptosis in murine leukemia cells of the L1210 line. (See also, Bilyy et al., In vivo expression and characteristics of novel α-D-mannose-rich glycoprotein markers of apoptotic cells. Cell Biology International 29 (2005) 920-928.) Although ConA also interacts with α-D-mannose, binding of this lectin by the apoptotic cells was insignificant. Such a result may be explained in part by the ability of ConA to bind not only α-D mannose but also α-D-glucose (Reeke G N Jr, Becker J W, Cunningham B A, et al. Relationships between the structure and activities of concanavalin A. Ann NY Acad Sci 1974;234:369-382). Thus, lectins specific for α-D-mannose and β-D-galactose may be used as specific markers of the apoptotic L1210 cells, which are used widely as the experimental cell model in studies of antitumor drug action (Golab J, Zagozdzon R, Kozar K, et al. Potentiated anti-tumor effectiveness of combined therapy with interleukin-12 and mitoxantrone of L1210 leukemia in vivo. Oncol Rep 2000;7:177-181). The relatively wide range of sugar specificity of WGA hinders its use for specific detection of apoptotic cells. Taking into account that lectins are more accessible and easier to use than monoclonal antibodies, this approach is a new and convenient tool for the detection of apoptotic cells.

### Example 3

Experiments with other cell types, specifically murine fibroblasts of the L929 line and human adenocarcinoma epithelial cells of the MCF7 line, also showed increased lectin binding with apoptotic cells as compared with normal cells. PSL (P<0.001), RCA-120 (P<0.01), and WGA (P<0.001) showed significantly stronger binding to apoptotic L929 cells, and RCA-120 (P<0.001) and VAA (P<0.001) demonstrated increased binding to apoptotic MCF7 cells, whereas the PSL effect was statistically insignificant (P=0.078). Thus, results of lectin cytochemical detection of apoptotic L1210 cells may be generalized to apoptotic cells of other types and species.

### Example 4

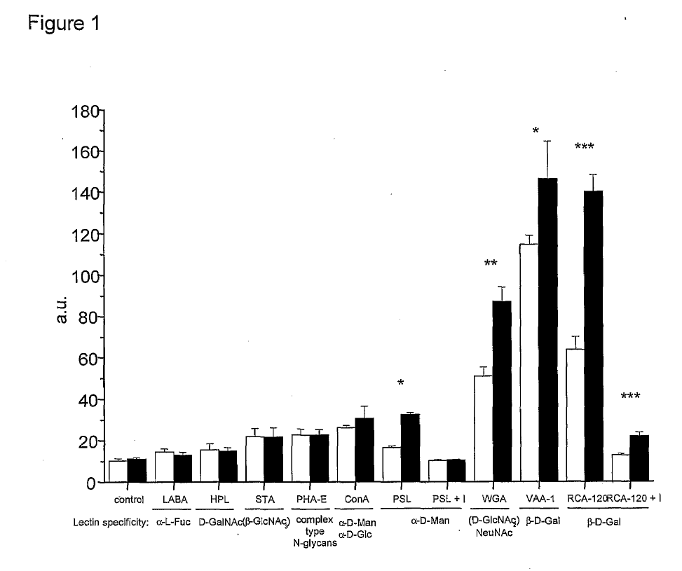

The inhibition of lectin binding by specific sugars and the use of different agents (methotrexate and cisplatin) for apoptosis induction indicate that increased HRP-lectin staining of the apoptotic cells mainly depends on specific lectin binding by plasma membrane glycoproteins expressed during apoptosis, and does not depend significantly on the nature of apoptosis-inducing agent. Decreased WGA binding has been reported in aneuploid multidrug-resistant variants of U87 and U373 cells in comparison with the intact cells of these lines (Camby I, Salmon I, Rombaut K, et al. Influence of culture media and multidrug resistance on the wheat germ agglutinin (WGA) glycocytochemical expression of two human glioblastoma cell lines. Anticancer Res 1996;16:1719-1725). However, it is believed that cisplatin resistance in L1210R cells does not depend on the multidrug-resistant mechanisms (Chu G. Cellular responses to cisplatin. Cancer 1994;269:787-790). It should be noted that the sugar specificity of WGA, RCA-120, and VAA binding is not absolute (Lutsik A D, Detjuk E S, Lutsik M D. Lectins in histochemistry [in Russian]. Lvov: Lvov University Press; 1989); thus, they can bind to glycoproteins possessing different carbohydrate moieties. This may explain the increased binding of WGA, RCA-120, and VAA with the non-apoptotic L1210 cells. VAA and RCA-120 are also very toxic for mammalian cells (Yakymovych M, Yakymovych I, Antonyuk V, et al. Lectins' cytotoxicity for L1210 murine leukemia cells with different sensitivity to anticancer drug cisplatin [in Ukranian]. Exp Physiol Biochem 1999;2:39-44). Concentrations of VAA, RCA-120, and ConA used in the lectin cytochemical studies were enough to demonstrate their cytotoxic effect and to induce apoptosis when added to culture medium of L1210 cells (Yakymovych M, Yakymovych I, Antonyuk V, et al. Lectins' cytotoxicity for L1210 murine leukemia cells with different sensitivity to anticancer drug cisplatin [in Ukranian]. Exp Physiol Biochem 1999;2:39-44; Lutsik M D. Antitumor properties of phytohemagglutinin from mistletoe. Proc Acad Sci Ukr SSR 1975;6:541-543). Interestingly, the level of cytotoxicity of VAA, RCA-120, and ConA (Stasyk T, Antonyuk V, Yakymovych M, et al. A comparative study of cell surface glycosyl determinants in cisplatin-sensitive and resistant L1210 murine leukemia cells. Exp Oncol 1998;20:204-209) correlated with the ability of these lectins to stain the apoptotic L1210 cells (FIG. 1). Strongly cytotoxic VAA and RCA-120 bound more intensively to the apoptotic cells, whereas ConA, which is relatively nontoxic for cells, was bound less intensively to these cells.

The PSL was bound by 32- and 49-kDa glycoproteins of L1210 cell membranes. In another study carried out in the inventors' laboratory (Stasyk T, Antonyuk V, Yakymovych M, et al. A comparative study of cell surface glycosyl determinants in cisplatin-sensitive and resistant L1210 murine leukemia cells. Exp Oncol 1998;20:204-209), binding of ConA and peanut Arachis sativum agglutinin specifically by 220- and 240-kDa glycoproteins were described in L1210 cells. These high-molecular-weight membrane receptors are of potential interest in studies of apoptosis in L1210 cells.

### Example 5

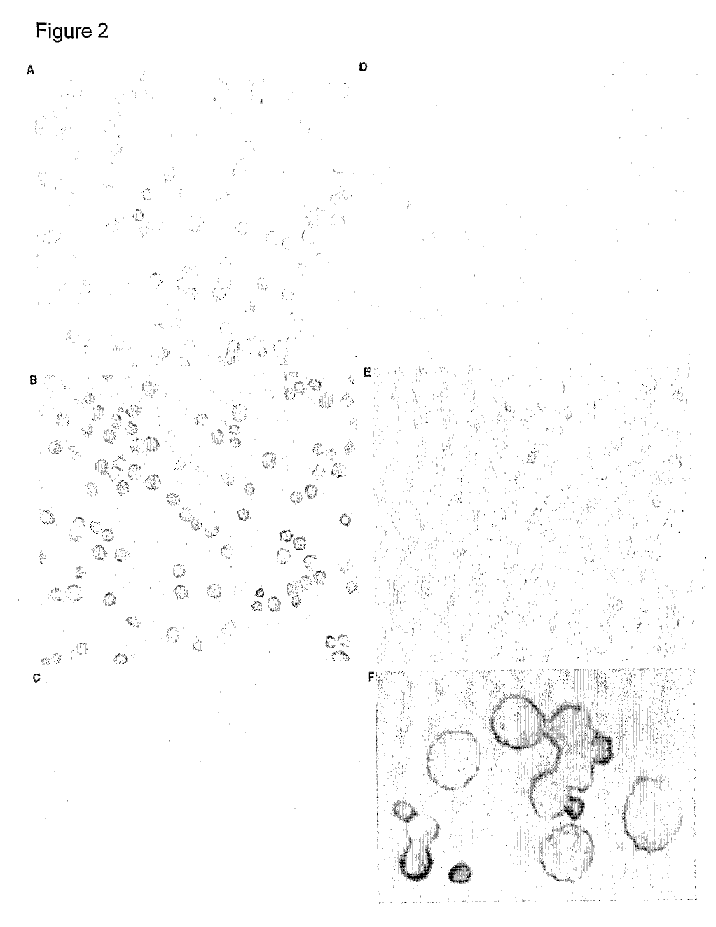

A different explanation for the increased expression of specific glycoproteins on the surface of apoptotic cells may be suggested. While not wishing to be bound to any specific theory, the inventors believe that it is related to the mechanisms of specific labeling of apoptotic cells and apoptotic bodies for their subsequent phagocytosis. Two studies found that phagocytosis of different pathogens is mediated via α-mannose and β-glucose receptors on the macrophages (Astarie-Dequeker C, N'Diaye E N, Le Cabec V, et al. The mannose receptor mediates uptake of pathogenic and nonpathogenic mycobacteria and bypasses bactericidal responses in human macrophages. Infect Immun 1999;67:469-477; Suzuki T, Ohno N, Ohshima Y, Yadomae T. Soluble mannan and beta-glucan inhibit the uptake of Malassezia furfur by human monocytic cell line, THP-1. FEMS Immunol Med Microbiol 1998;21:223-230). The addition of galactose, acetyl-D-galactosamine, N-acetyl-D-glucosamine, mannose, and αMMan inhibited ingestion of pathogenic bacteria by the polymorphonuclear leukocytes (Register K B, Ackermann M R, Kehrli M E Jr. Non-opsonic attachment of Bordetella bronchiseptica mediated by CD1/CD18 and cell surface carbohydrates. Microb Pathog 1994;17:375-385). Amino sugars such as glucosamine, N-acetyl-glucosamine, and galactosamine inhibited uptake of apoptotic eosinophils by resting and interleukin-1α-stimulated small airway epithelial cells (Walsh G M, Sexton D W, Blaylock M G, Convery C M. Resting and cytokine-stimulated human small airway epithelial cells recognize and engulf apoptotic eosinophils. Blood 1999; 94:2827-2835). Mannose receptors (175-kDa surface C-type lectin) of macrophages, dendritic cells, sinus-lining cells of the spleen, and lymph nodes may be very important in the removal of aged cells and the phagocytosis of mannose-coated particles (Uccini S, Sirianni M C, Vincenzi L, et al. Kaposi's sarcoma cells express the macrophage-associated antigen mannose receptor and develop in peripheral blood cultures of Kaposi's sarcoma patients. Am J Pathol 1997; 150:929-938). Phagocytosis by human neutrophils of ConA-treated erythrocytes and non-opsonized Escherichia coli cells involves mannose-binding adhesions mediated by the Fc γ receptor (Salmon J E, Kapur S, Kimberly R P. Opsonin-independent ligation of Fc gamma receptors. The 3G8-bearing receptors on neutrophils mediate the phagocytosis of concanavalin A-treated erythrocytes and nonopsonized Escherichia coli. J Exp Med 1987;166:1798-1813; Salmon J E, Kimberly R P. Phagocytosis of concanavalin A-treated erythrocytes is mediated by the Fc gamma receptor. J Immunol 1986;137:456-462). Thus, increased expression of mannose- and galactose-rich glycoproteins on the surface of apoptotic cells may be important for the phagocytosis of these cells and apoptotic bodies. This is in accordance with the appearance of the higher density of mannose-rich glycoproteins on the surface of apoptotic bodies shown during the lectin cytochemical analysis using PSL binding by apoptotic cells (FIG. 2F).

### Example 6

The data presented show that an increase in expression of membrane glycoproteins containing α-D-mannose and β-D-galactose positively correlates with the incidence of apoptosis in the studied cells (L929, L1210, MCF-7 (wt), MCF-7 (DOX/R), and Jurkat cell lines). This was demonstrated by an increased bidding of mannose-specific lectins (PSL, GNA, PMRL, NPA, LVA) and galactose-specific lectins (RCA, VAA) with apoptotic cells compared to their binding with non-apoptotic cells, Lectinocytochemical analysis of intact and apoptotic cells also showed an increase in binding of WGA lectin to apoptotic cells, although this lectin possesses a wide range of carbohydrate specificity, for example, (D-GlcNAc)ₙ, where n=1, 2, 3, and NeuNAc (Lutsik et al. 1981). Taking into account that these carbohydrate residues are wide spread in the glycocalyx, the use of WGA may not be a good choice for apoptosis detection. VAA lectin binding to the apoptotic cells in some cases was significantly stronger than to non-apoptotic cells; however, in other cases that difference was not reliable within an assumed level of significance at 0.05.

In addition to 13 lectins widely used in the histochemical analysis, 2 lectins (PMRL and LVA), which are used less frequently, were isolated in the inventors' laboratory (Antoniuk L, Antoniuk V. Interaction of immobilized lectin from Leucojum vernum L. with polysaccharides and glycoproteins. Ukr Biokhim Zh. 1993; 65: 69-76. [in Ukrainian]; Antoniuk V. Purification and properties of lectins of Polygonatum multiflorum [L.] All. and Polygonatum verticillatum [L.] All. Ukr Biokhim Zh. 1993; 65: 41-48 [in Ukrainian]) and tested as an instrument for discriminating between the apoptotic and non-apoptotic cells. One of them, namely PMRL, was an effective marker of the apoptotic cells, and was important in studies of apoptosis using cell agglutination test. The level of binding of other lectins, such as LABA, HPL, STA, PHA-E, ConA, and LVA, with non-apoptotic and apoptotic cells was found to be similar. While not wishing to be bound to any particular theory, the inventors believe that only the expression of specific types of glycoproteins is altered during apoptosis. As an exception, an increased binding of HPL with apoptotic Jurkat cells can be noted. One may speculate about the role of blood group antigens expression in Jurkat cells, to which HPL possess high affinity (blood group typing) (Khan F, Khan R, Sherwani A, Mohmood S, Azfer M. Lectins as markers for blood grouping. Med Sci Monit 2002; 8: 293-300). It should be noted that the specificity of all lectinocytochemical reactions was controlled by inhibition of lectin binding by specific sugars.

### Example 7

The inventors found that an increased expression of plasma membrane α-D-mannose- and β-D-galactose-containing glycoproteins at apoptosis did not depend on type of used cell line, as well as on nature of apoptosis-inducing agent—chemical (cisplatin, methotrexate, dexamethasone, etoposide) or physical (X-radiation, hyperthermia). An increased expression of apoptosis-dependent cell membrane glycoproteins was also independent of the way of cell detachment—trypsinization which may potentially impair some plasma membrane proteins, or mechanical rubbing which may act in different way. An increase in membrane glycoprotein expression revealed by means of the lectinocytochemical analysis, exhibited dependence on concentration of apoptosis-inducing agent. That increase may be clearly detected as early as 12 hours after cell treatment with apoptosis-inducing agents. Other manners of cell fixation (fixation in formalin vapors and utilization of unfixed cells) had no significant effect on cells' ability to bind PSL and RCA lectins, thus excluding possible effect of redistribution of intracellular carbohydrate moieties during the fixation procedure.

### Example 8

To address the question on the origin of glycoprotein(s) expressed on the apoptotic cells, while not wishing to be bound to any particular theory, the inventors believe that it is due to a modification of the pre-existing glycoproteins, rather than their synthesis de novo or redistribution within the target cells. It was demonstrated that apoptosis caused by nitric oxide donors in the sublingual salivary gland acinar cells in culture was accompanied by a decrease in glycoprotein synthesis (Slomiany B L, Slomiany A. Nitric oxide interferes with salivary mucin synthesis: involvement of ERK and p38 mitogen-activated protein kinase. J Physiol Pharmacol. 2002; 53: 325-336). Lysosomal (sialidase) (Neul) activity was shown to be elevated after apoptosis induction by sodium butyrate in human colon cancer cells (Kakugawa Y, Wada T, Yamaguchi K, Yamanami H, Ouchi K, Sato I, Miyagi T. Up-regulation of plasma membrane-associated ganglioside sialidase (Neu3) in human colon cancer and its involvement in apoptosis suppression. Proc Natl Acad Sci U S A. 2002; 99: 10718-23). Azuma et al. reported about an increase in RCA binding to apoptotic Jurkat cells caused by their exposure to neuraminidase whose activity was induced by etoposide treatment (Azuma Y, Taniguchi A, Matsumoto K. Decrease in cell surface sialic acid in etoposide-treated Jurkat cells and the role of cell surface sialidase. Glycoconj J. 2000; 17: 301-306). Hydrolysis of sialic acids by the sialidase was dominant in tumor cell apoptotic bodies. This is considered to be important for better recognition of apoptotic bodies by C-type lectins present in macrophages which engulf the apoptotic bodies (Uehara F, Ohba N, Miyagi T. Glycohistochemical analysis of apoptotic bodies in eyelid tumor. Nippon Ganka Gakkai Zasshi. 1997; 101: 611-6). Further experiments, conducted by the inventors provide evidence that the inhibitors of de novo glycopoteins synthesis (tunicamycin, 2-deoxy-D-glucose) and the inhibitor of glycoprotein traffic from Golgi to plasma membrane (monensine) did not diminish an increase in the expression of α-D-mannose- and β-D-galactose-containing glycoprotein(s) in the apoptotic cells. It was also shown that apoptosis in the target cells was accompanied by a 40-fold increase in the membrane-associated neuraminidase activity. Such activity was not detected in the conditioned medium after cell cultivation or apoptosis induction. An artificial desialation of plasma membrane glycoproteins, performed by acid methanolysis, led to an increase in the levels of α-D-mannose- and β-D-galactose-containing glycoprotein(s). Thus, the available data show that during apoptosis, plasma membrane glycoproteins can be modified via desialation caused by an activation of membrane-associated neuraminidase activity. It is known that neuraminidase activity may lead to the exposure of galactose- and/or mannose-residues which are recognized by RCA, VAA, PSL, and GNA lectins used in the inventors' study.

Pretreatment of cells for 2 hours with non-labeled lectin decreased plasma membrane staining with corresponding HRP-labeled lectin, which may be explained by lectin-induced internalization of specific membrane glycoproteins. It was reported by Liu et a/. that fluorescein-conjugated WGA was transported from the cell surface into the paranuclear region of cultured L929 cells within 30 min where it affected some signaling pathways with resulting arrest of cell cycle (Liu W, Sze S, Ho J, Liu B, Yu M. Wheat germ lectin induces G2/M arrest in mouse L929 fibroblasts. J Cell Biochem. 2004; 91: 1159-1173). Internalization of the lectin receptors may also suggest their involvement in some signaling pathways.

### Example 9

Comparison of lectin-induced agglutination in the non-apoptotic and apoptotic cells showed that such testing can be another simple and reliable method for detecting changes in specific glycoprotein expression in the apoptotic cells and even used for their semi-quantitative detection. The inventors found that lectin (PSL) concentration, needed to aggiutinuate non-apoptotic cells was eight times higher than that needed for the apoptotic cells. The inventors believe that PSL is not the only lectin which may discriminate between non-apoptotic and apoptotic cells in the agglutination test. It should be stressed that the agglutination test is much simpler than the lectinocytochemical analysis, as it does not require lectin labeling and computer densitometry of HRP-lectin-stained cells. Bilyy et al., (Some New Approaches to the Detection of Programmed Cell Death. Proc. of SPIE Vol. 6163 61630J-1, Fall 2005) provides a survey of approaches to the detection of apoptosis, including methods described by the present invention. Agglutination studies comparing apoptotic and intact cells have confirmed that lectin binding receptors are located in the plasma membrane of non-fixed cells, and their appearance were not caused by the exposure of intracellular lectin binding sugar moieties which could potentially appear on the cell surface during cell fixation procedure.

### Example 10

The inventors demonstrated that lectin-conjugated agarose may be utilized for isolation of population of apoptotic cells. It should be noted that duration and temperature of cell incubation with lectin-conjugated agarose beads had a crucial effect on the apoptotic cell isolation. To the inventors' knowledge, this is the first example of such approach in studying the apoptotic cells.

Despite a similar increase in α-D-mannose- and β-D-galactose-containing glycoprotein expression in the apoptotic cells of different studied cell lines, densitometric profiles at their lectinocytochemical analysis were not identical.

Lectins which were discovered more than 100 years ago, recently found new application as novel markers of different types and subtypes of cells, e.g. -GNA and N. pseudonarcissus lectins were shown to bind specifically with macrophages, WGA (succinylated)—with type I pneumocytes (Barkhordari A, Stoddart R W, McClure S F, McClure J. Lectin histochemistry of normal human lung. J Mol Histol. 2004; 35(2):147-56), Amaranthus leucocarpus lectin—with naive T cells (Porras F, Lascurain R, Chavez R, Ortiz B, Hernandez P, Debray H, Zenteno E Isolation of the receptor for Amaranthus leucocarpus lectin from murine naive thymocytes. Glycobiology 2000; 10: 459-465). Different lectins were also used as markers for specific tumour determinants (Guillot J, Guerry M, Konska G, Caldefie-Chezet F, De Latour M, Penault-Llorca F. Modification of glycoconjugates during the carcinogenesis: the case of mammary carcinomas. Bull Cancer. 2004; 91:141-58. [inFrench]; Wu A. Polyvalency of Tn (GalNAcalpha1->Ser/Thr) glycotope as a critical factor for Vicia villosa B(4) and glycoprotein interactions. FEBS Lett. 2004; 562: 51-8).

The inventors believe that biological role of elevated expression of α-D-Man- and β-D-Gal-containing glycoproteins during apoptosis could be important for labeling the apoptotic cell for their next elimination by the macrophages and/or neighboring epithelial cells. An ability of the immunocompetent cells to use lectin receptors for interaction with glycoproteins on targeted cells may support such suggestion (Geijtenbeek T, Van Vliet S, Engering A, T Hart B, Van Kooyk Y Self- and Nonself-Recognition by C-Type Lectins on Dendritic Cells. Annu Rev Immunol 2004; 22: 33-54; Van De Wetering J, Van Golde L, Batenburg J Collectins. Eur J Biochem. 2004; 271: 1229-1249; Nauta A, Raaschou-Jensen N, Roos A, Daha M, Madsen H, Borrias-Essers M, Ryder L, Koch C, Garred P. Mannose-binding lectin engagement with late apoptotic and necrotic cells Eur. J. Immunol. 2003; 33: 2853-2863; Pittoni V, Valesini G The clearance of apoptotic cells: implications for autoimmunity. Autoimmun Rev. 2002; 1: 154-161).

### Example 11

### Cells

Two sublines of murine leukemic cells of the L1210 line, cisplatin-sensitive (L1210) and -resistant (L1210R), were obtained from the Cell Culture Collection of the R. E. Kavetsky Institute of Experimental Pathology, Oncology and Radiobiology, National Academy of Sciences of Ukraine (Kyiv, Ukraine). Cells were maintained in a suspension culture consisting of Dulbecco's Minimum Essential Medium (Sigma Chemical Co., St. Louis, Mo.) supplemented with 10% heat-inactivated fetal calf serum (Sigma Chemical Co.) and gentamicin (50 ∥g/ml; Sigma Chemical Co.). Methotrexate (100 ∥g/ml; Lederle Parenterars, Carolina, P R) or cisplatin (0.5 or 5 μg/ml; Ebewe, Austria) was used for apoptosis induction.

Additionally, murine leukemic cells of L1210 line, murine fibroblasts of L929 cell line, human adenocarcinoma MCF-7 line wild type (wt) and resistant to doxorubicin (DOX/R), human leukemia Jurkat cell lines were obtained from the Cell Culture Collection of Institute of Cell Biology, National Academy of Sciences of Ukraine (Lviv, Ukraine). L1210, L929 and MCF-7 cells lines were maintained in DME medium (Sigma Chemical Co., USA), and the Jurkat cell line was maintained in RPMI 1640 medium (Sigma Chemical Co., St. Louis, USA); culture medium was supplemented with 10% heat-inactivated fetal calf serum (Sigma) and gentamycin (50 μg/ml, Sigma). Apoptosis of L1210 cells was induced by cisplatin (0.5 and 0.1 μg/ml, or 5.0 μg/ml, Ebewe, Austria) (56,57); apoptosis of L929 cells was induced by hyperthermia 43° C. (Tomasovic S, Vasey T, Story M, Stephens L, Klostergaard J. Cytotoxic manifestations of the interaction between hyperthermia and TNF: DNA fragmentation. Int J Hyperthermia. 1994; 10: 247-262; Yuen W, Fung K, Lee C, Choy Y, Kong S, Ko S, Kwok T. Hyperthermia and tumour necrosis factor-alpha induced apoptosis via mitochondrial damage. Life Sci 2000; 67: 725-732) and etoposide (10 μM, Bristol-Myers, USA) (Karpinich N, Tafani M, Rothman R, Russo M. The course of etoposide-induced apoptosis from damage to DNA and p53 activation to mitochondrial release of cytochrome c. JBC. 2002; 277: 16547-16552); apoptosis of MCF-7 line was induced by methotrexate (100 μg/ml, Lederle Parenterars, Carolina, Puerto Rico) (Ruiz-Ruiz M, Lopez-Rivas A. p53-mediated up-regulation of CD95 is not involved in genotoxic drug-induced apoptosis of human breast tumor cells. Cell Death Differ. 1999; 6: 271-280) or by X-radiation (3.0 Gy, 2.25 Gy/min), followed by 24 h recovery period) (Ding et al. 2001); apoptosis of Jurkat cells was induced by cisplatin (5 μg/ml) (Guchelaar H, Vermes I, Koopmans R, Reutelingsperger C, Haanen C. Apoptosis- and necrosis-inducing potential of cladribine, cytarabine, cisplatin, and 5-fluorouracil in vitro: a quantitative pharmacodynamic model. Cancer Chemother Pharmacol. 1998; 42: 77-83) or by dexamethasone (dexamethasone phosphate, 1 μM, Lvivdialik, Lviv, Ukraine) (Strauss G, Osen W, Debatin K M. Induction of apoptosis and modulation of activation and effector function in T cells by immunosuppressive drugs. Clin Exp Immunol. 2002; 128: 255-266). Lymphocytes of healthy donors and patients with autoimmune disorders were isolated using LympoPrep (Nikomed Pharma AS, Norway) according to the manufacture's instructions at the Department of Immunology and Allergology of Lviv National Medical University (Ukraine). Cell viability was controlled by trypan-blue (0.1% w/v solution) exclusion test, and cells were counted in hemocytometric chamber under light microscope.

### Example 12

### Lectins

The following plant lectins were used in the experiments: Labumum anagyroides bark agglutinin (LABA), Phaseolus vulgaris agglutinin (PHA-E), PSL, RCA-120, Solanum tuberosum agglutinin (STA), Triticum vulgaris agglutinin (wheat germ agglutinin, WGA), Viscum album agglutinin (VAA), Canavalia ensiformis lectin (concanavalin A, ConA), Helix pomatia lectin (HPL), Maackia amurensis lectin II (MAL-II), LABA, GNA, PMRL, NPA and LVA. All lectins except ConA (Lectinola, Czech Republic) and MAL-II (Vector laboratories, USA) were isolated and purified to electrophoretic homogeneity in the inventors' laboratory, as previously described (Khopade A J, Nandakumar K S, Jain N K. Lectin-functionalized multiple emulsions for improved cancer therapy. J Drug Target 1998;6:285-292). Lectins were labeled by horseradish peroxidase (HRP), biotin or fluorescein isothiocyanate (FITC). α-Methyl-D-mannopyranoside (αMMan; Sigma Chemical Co.) and 4-O-(α-D-galactopyranosyl)-D-glucopyranose (lactose; Sigma Chemical Co.) were used as sugar inhibitors of PSL and RCA-120 binding, respectively (19).The following lectins were also used in the experiments: LABA, PHA-E, PSL, RCA, STA, WGA, VM, ConA, HPL, GNA, PMRL and LVA. Lectins (electrophoretic homogeneity) were purchased from Lectinotest Laboratory (Lviv, Ukraine). ConA was produced by Lectinola (Czech Republic). For lectinocytochemical studies, lectins were labeled by HRP, and for agglutination analysis, non-labeled lectins were used.

The following lectins were also used in the experiments VAA, SNA, PMRL, LLA, PLA, L-fucose specific lectin from river perch, HPL, RCA, PSL to estimate their ability to agglutinate intact and apoptotic cells.

### Example 13

### Lectin Cytochemistry

Lectin cytochemical analysis was conducted as described previously by Herrington & Mc Gee (Herrington C S, McGee JO'D. Diagnostic molecular pathology. Oxford: IRL Press; 1992), with some modifications. Cell smears were fixed in a mixture of acetone, methanol, and formalin (19:19:2) for 90 s at room temperature and then air dried. Smears were washed twice with Tris saline buffer (TSB) for 2 min and incubated with HRP-labeled lectins (50 μg/ml) for 1 h at room temperature or overnight at 4° C. If necessary, the appropriate sugar inhibitor (0.1 M solution) was added to the incubation mixture. Smears were washed twice with TSB for 10 min and incubated with 0.5 mg/ml of 3,3′-diaminobenzidin (Sigma Chemical Co.) and 4 μl/ml of H₂O₂ in TSB for 5 min. In some experiments a NiCl₂ solution was added to the incubation mixture (final concentration, 1 mg/ml) to improve cell contrast.

Smears were washed in distilled water, air dried, and mounted in Canadian balsam. Densitometry of mounted smears was conducted by using images obtained by a Biolam microscope (Lomo, St. Petersburg, Russia) equipped with a video-capturing device. Densitometric analysis was performed on an IBM computer running PhotoM 1.21 and UTHSCSA ImageTool, which was developed at the University of Texas Health Science Center at San Antonio.

Alternatively, smears were washed in distilled water, air dried and photographed. Densitometric analysis was performed on an AMD-based IBM PC computer using ImageJ (Wayne Rasband, National Institutes of Health, USA) program support and the UTHSCSA ImageTool program (University of Texas Health Science Center in San Antonio, Tex.).

### Example 14

### Lectinoblotting

For isolation of membrane fractions, L1210 cells were washed and then suspended in hypotonic buffer (10 mM Tris-HCl, pH 7.5; 1.5 mM MgCl₂, 1 mM phenyl methyl sulfonyl fluoride, and 1 mM benzamidine, protease inhibitor cocktail, Sigma, was added according to manufacturer's instructions), kept for 10 min at 0° C., and then homogenized with a Potter homogenizer. An appropriate volume of 2 M sucrose was added immediately to the homogenate to achieve a final concentration of 0.25 M, and the suspension was centrifuged for 15 min at 2,000 g for pelleting nuclei and intact cells. The pellet was homogenized once more in the hypotonic buffer. Supernatants of three homogenizations were combined and centrifuged for 60 min at 25,000 g. All operations were performed at 4° C. Electrophoresis was carried out in 5% to 17.3% gradient PAAG using the Laemmli buffer system (Laemmli UK. Cleavage of structural proteins during the assembly of the head of bacteriophage T4. Nature 1970;277:680-685). Membrane proteins were electrophoretically transferred onto nitrocellulose sheets (0.45 μm; type HA, Millipore, Bedford, Mass.), or PVDF membrane (BDH Lab Supplies, U.K.), as described previously (Towbin M, Stehelin T, Gordon I. Electrophoretic transfer of protein from polyacrylamide gels to nitrocellulose sheets: procedure and some applications. Proc Natl Acad Sci USA 1979;76:4350-4354). Glycoproteins were demonstrated on blots by using HRP-labeled lectins, as described previously (Lutsik M D, Kusen S I. A study of membrane glycoproteins of human erythrocytes with use of lectins [in Russian]. Ukr Biochem J 1987; 59:3-9).

### Example 15

### DNA Preparation and Electrophoresis

Briefly, 5×10⁶ cells were pelleted and resuspended in 50 μl of 20 mM ethylene-diamine-tetraacetic acid plus 50 mM Tris-HCl, pH 7.5, and centrifuged for 5 min at 1,600 g, and pellets were resuspended in lysis buffer. Sodium dodecyl sulfate (final concentration, 1%) and RNase A (final concentration, 1 mg/ml; Sigma Chemical Co.) were added to each sample, which were then incubated for 1 h at 37° C. Thereafter, proteinase K (final concentration, 1 mg/ml; Boehringer Mannheim, Mannheim, Germany) was added to each sample, which was then incubated for 1 h at 37° C. Then 10 M ammonia acetate (50% of the sample volume) was added to each sample, and DNA was precipitated with 2 vol of ice-cold isopropanol overnight at −20° C. Samples were centrifuged for 30 min at 10,000 g, and pellets were air dried, dissolved in TE buffer (10 μl/10⁶ cells), and loaded into the dry wells of 1% (w/v) agarose gel. Electrophoresis was carried out in 1 mM ethilene-diamine-tetraacetic acid plus 40 mM Tris-acetate buffer, pH 8.0, until the marker dye migrated 6 to 7 cm. Electrophoregrams were stained with ethidium bromide, screened in a transilluminator under ultraviolet light, and photographed.

### Example 16

### Statistical Analysis

Experiments were performed in triplicate and repeated three times. Statistically significant differences in a typical experiment were assessed by Student's t-test. The level of significance was set at 0.05. Statistical interpretation of the densitometric data was done with Microcal Origin (Microcal Software, Northampton, Mass.).

### Example 17

### Agglutination

A 20 μl of cell suspension with 5×10⁶ cells/ml were added to 20 μl of lectin solutions (dilutions from 10,000 to 10 μg/ml) in agglutination tube and centrifugated at 200 g for 60 s. Mixtures were resuspended once and 10 μl were transferred on slide glass, and examined under microscope.

Alternatively, 20 μl of cell suspension with 10⁷ cells/ml were added to 20 μl of lectin solutions (dilutions from 1,000 μg/ml to 7.8 μg/ml) in 96-well immunological plate and incubated for 30 min at room temperature. Agglutinates were scanned using Epson transmissive scanner using 3,200 dpi resolution.

This agglutination method led to a considerable simplification of the procedure, a 10-fold decrease in lectin amount needed for 1 analysis, and provided an ability to directly document the results using scanning in a “transparency” mode.

### Example 18

### Cell Separation

The cell suspension (10⁶ cells/ml) was washed twice with TSB, pH 7.4 and 2 ml of the suspension were added to 2 ml of PSL-conjugated coarse-grained agarose (4.5 mg of PSL protein per ml of agarose) and incubated in 35 mm plastic Petri dish at 37° C. for 30 min. Then the suspension was transferred to a column with inert metal sieve that allowed passing of cells but retarded agarose particles in the bottom. The diameter of column was chosen in such a way that agarose layer did not exceed 24 mm. Column was washed with double volume of TSB, pH 7.4, and the fraction of unbound cells was collected. Then the column was washed with double volume of 0.05 M borate buffer, pH 8.0, and the fraction of PSL-bound cells was collected. Cells were then washed with TSB or PBS buffer, concentrated by centrifugation and used for further study (See FIG. 12A).

### Example 19

### Fluorescent Microscopy

Acridine orange (final concentration 1 μg/ml, Sigma) was added to the cell suspension for 30 min. Cells were examined under LUMAM-P2 fluorescent microscope (LOMO, USSR) and photographed.

### Example 20

### Binding of Different Lectins to Intact and Apoptotic L1210 Cells

Examination of the of binding of different HRP-labeled lectins (LABA, HPL, STA, PHA-E, ConA, PSL, WGA, VAA, and RCA-120) with plasma membrane components of normal and apoptotic L1210 cells showed significant differences between the densitometric profiles of binding by the normal and apoptotic cells (apoptosis was induced by 100 μg/ml of methotrexate; FIG. 1). Apoptotic cells were stained more 21 (P<0.05), WGA (P<0.01), VAA (P<0.05), and RCA-120 (P<0.001) than were intact cells. RCA-120, WGA, and VAA stained apoptotic and intact cells, whereas other lectins did not stain or weakly stained the intact cells. There were no differences between normal and apoptotic cells with respect to their ability to bind LABA, HPL, STA, PHA-E, and ConA. Further, these lectins were only slightly bound by the intact cells. Specific sugar inhibitors of lectin binding, such as α-MMan for PSL and lactose for RCA-120, showed substantial (for RCA-120) or almost absolute abolishment of lectin binding (FIG. 1). Microscopic examination of cell smears showed uniform distribution of glycosylated components on the plasma membrane in the case of binding of all used lectins except PSL. Binding of HRP-labeled PSL showed more intensive accumulation of binding components by the vesicles observed on the cell surface. These vesicles may represent the visible apoptotic bodies (FIG. 2F).

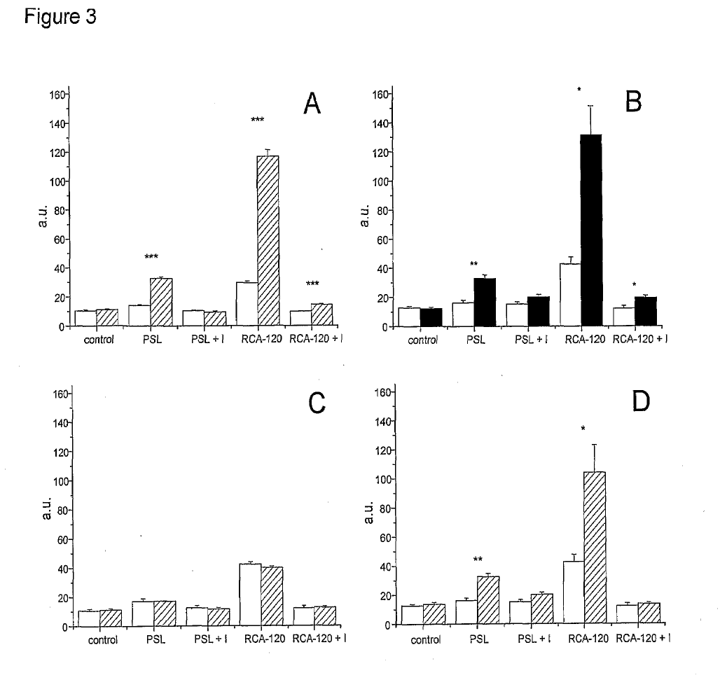

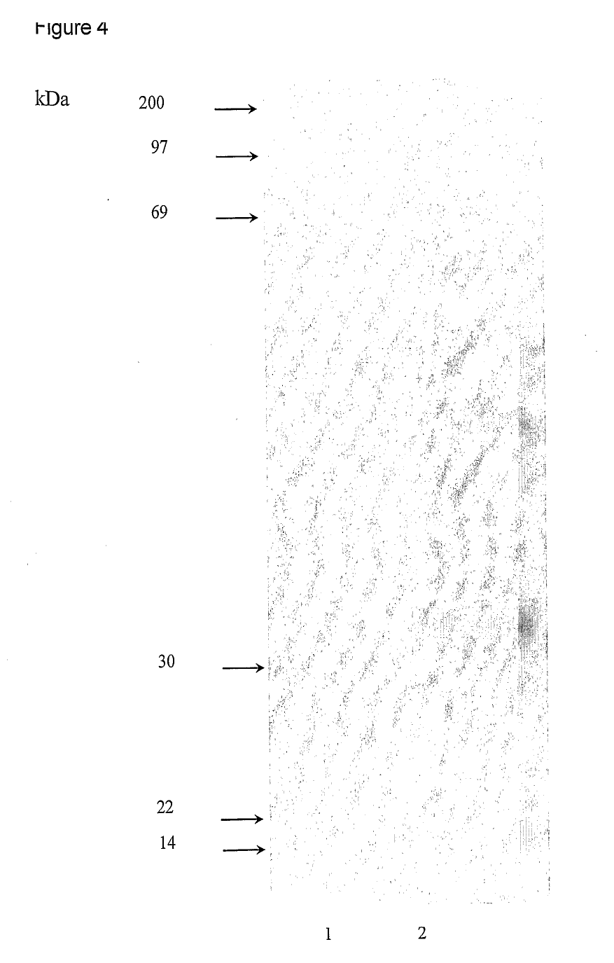

Apoptosis induction by cis-diamminodichloroplatinum (cisplatin; 0.5 μg/ml) showed changes in cell densitometric characteristics similar to those induced by methotrexate (FIG. 3). RCA-120 and PSL were much better bound by the apoptotic cells (FIG. 2B and 2E) than by the normal ones (FIG. 2A and 2D; P<0.001). In the presence of specific sugars inhibiting these lectins binding, no or very insignificant lectin binding was observed (FIG. 2C). PMG sodium dodecyl sulfate electrophoresis and lectin blotting showed that the receptors for the PSL ligand were present in the membrane fraction and absent in the soluble fraction (cytoplasm; FIG. 4). Two glycoproteins (molecular weights of 32 and 49 kDa) binding PSL were predominantly expressed.

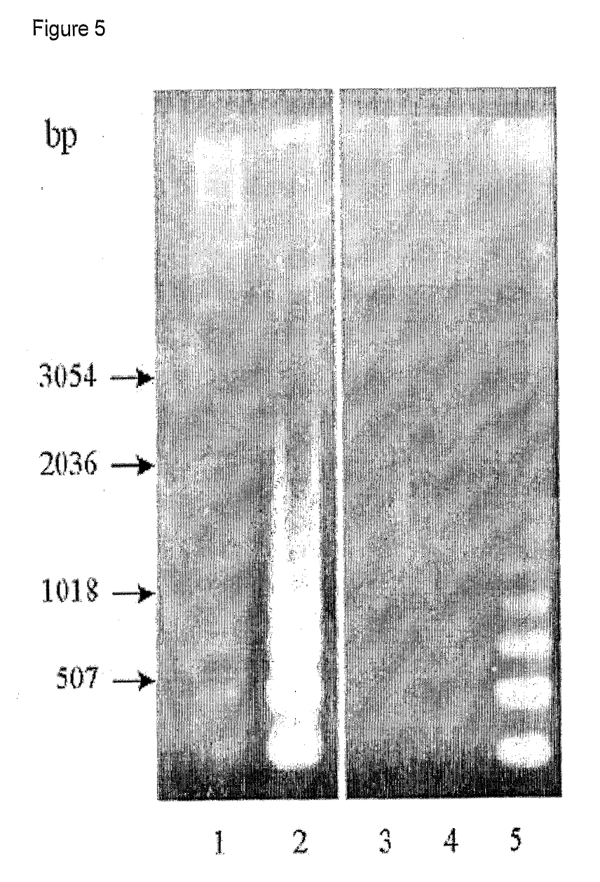

To determine whether changes in the expression of PSL receptors were specific for the apoptotic cells, the inventors also used a sub-line of L1210R cells resistant to the action of cisplatin. The cisplatin concentration (0.5 μg/ml) that did not induce apoptosis in these resistant cells (FIG. 5) also did not change the expression of their specific membrane glycoproteins (FIG. 3C). Nevertheless, the higher cisplatin concentration (5 μg/ml) that induced apoptosis in L1210R cells affected glycoprotein expression in a manner similar to that of methotrexate (100 μg/ml), which also induced apoptosis in these cells (FIG. 3D). Densitometric characteristics of PSL (P<0.01) and RCA-120 (P<0.05) binding by the apoptotic L1210R cells resembled the characteristics of the apoptotic L1210S cells.

### Example 21

### Lectinocytochemical Study of Apoptotic Cells of Different Lines

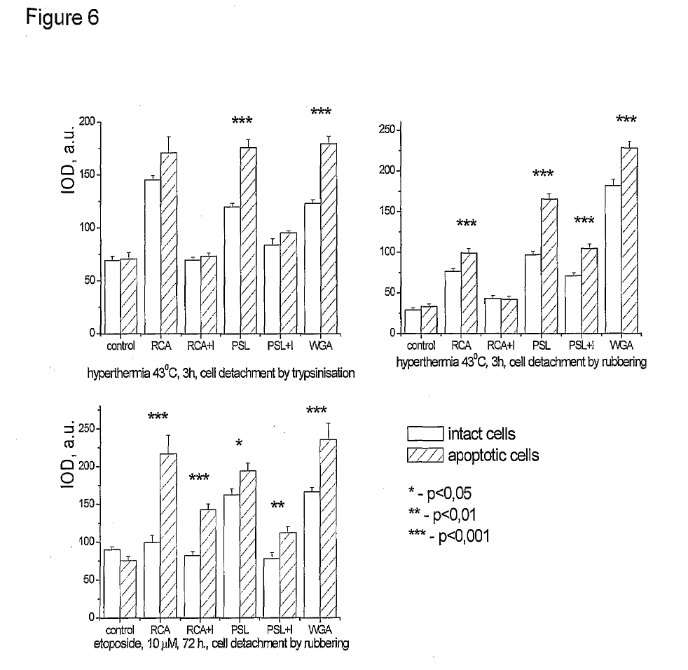

Using of HRP-labeled LABA, PHA-E, PSL, RCA, STA, WGA, VAA, ConA, and HPL lectins for lectinocytochemical analysis of non-apoptotic and apoptotic (hyperthermia, 43° C., 3h) transformed murine fibroblasts of L929 line revealed increased binding of PSL (p<0.001) and WGA lectins (p<0.001) by the apoptotic cells compared to the non-apoptotic cells detached by rubbing. Similar binding patterns were observed when trypsinization was used for cellular detachment from culture dish: the apoptotic cells bound PSL, WGA and RCA significantly stronger (p<0.001) than the intact cells. Apoptosis induction by etoposide (10 μM, 72 h) demonstrated changes in cell binding characteristics close to those induced by hyperthermia. There was more intensive staining of the apoptotic cells by HRP-labeled PSL (p<0.05), RCA (p<0.001), WGA (p<0.001) and VAA lectins (p<0.05) in comparison with such staining of the non-apoptotic cells (FIG. 6). No differences were observed between non-apoptotic and apoptotic cells in their ability to bind LABA, HPL, STA, PHA-E, and ConA. In controls, buffer was used instead of HRP-labeled lectin. Specific sugar inhibitors, such as α-methyl-mannoside (α-MMan) and 4-O-(α-D-galactopyranosyl)-D-glucopyranose (lactose), were also added in the cases of PSL and RCA utilization, respectively.

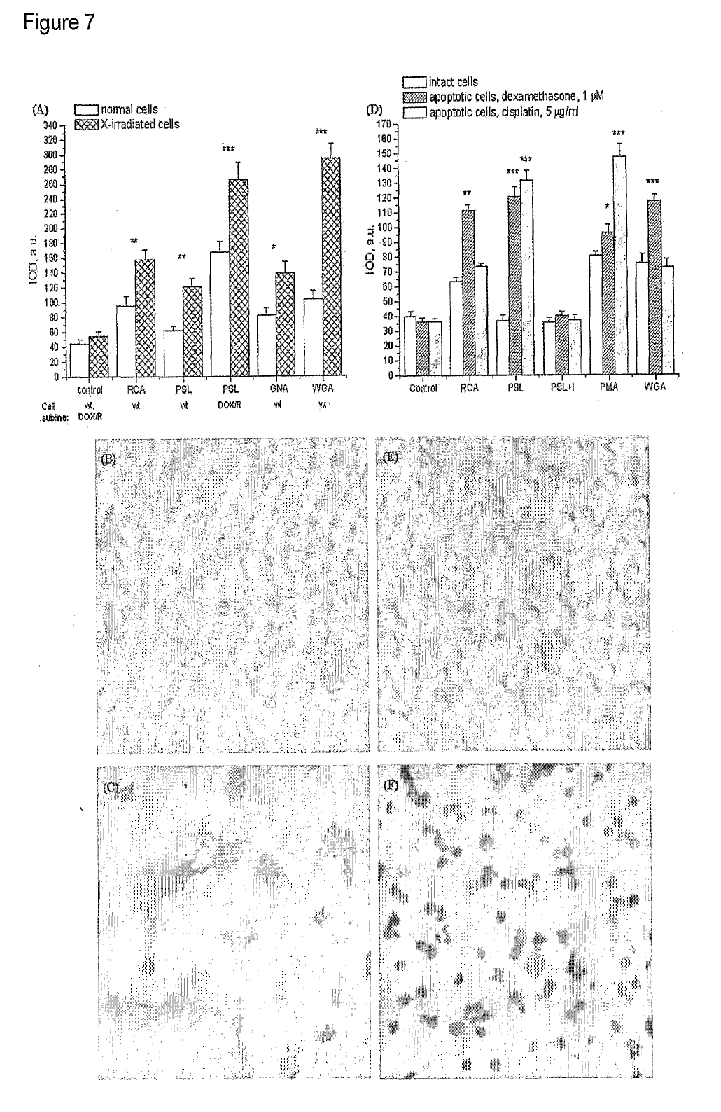

Lectinocytochemical analysis was applied for two sublines of human adenocarcinoma cells of MCF-7 line—wt and DOX/R. Densitometric study demonstrated similar characteristics of apoptotic cells with an increase in binding α-D-mannose specific lectins (PSL, p<0.01, and GNA, p<0.05) and β-D-galactose-specific lectins (RCA, p<0.05, and VAA, p<0.001 in the case of methotrexate action), as well as (D-Glc-NAc)ₙ- and NeuNAc-specific WGA lectin (p<0.001), to the apoptotic cells compared to the non-apoptotic ones. These changes were observed for both the induction of apoptosis by methotrexate (100 μg/ml, 24 h) and by X-ray radiation (300 R-units, 225 R-units/min, followed by 24 h recovery period) (FIGS. 7A-C).

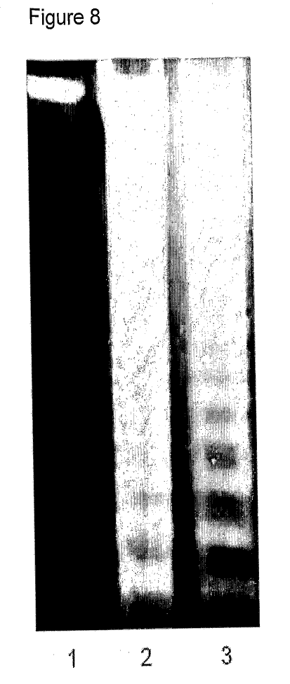

Binding of PSL (p<0.001), PMRL (p<0.05 in the case of dexamethasone action (1 μM, 24 h) and p<0.001 in the case of ciplatin action (5 μg/ml, 24 h), GNA (p<0.01 in the case of cisplatin action), RCA (p<0.001 for dexamethasone action and p<0.05 for cisplatin), and VAA (p<0.05 for cisplatin action) lectins were stronger with apoptotic human leukemia Jurkat cells, than with non-apoptotic cells of this line (FIGS. 7D-F). Appearance of apoptosis was controlled by DNA laddering (FIG. 8). HPL lectin also bound more strongly to apoptotic Jurkat cells than to non-apoptotic cells (p<0.05).

### Example 22

### Dose-Dependent Effect of Apoptosis Inducing Agents on Glycoprotein Expression

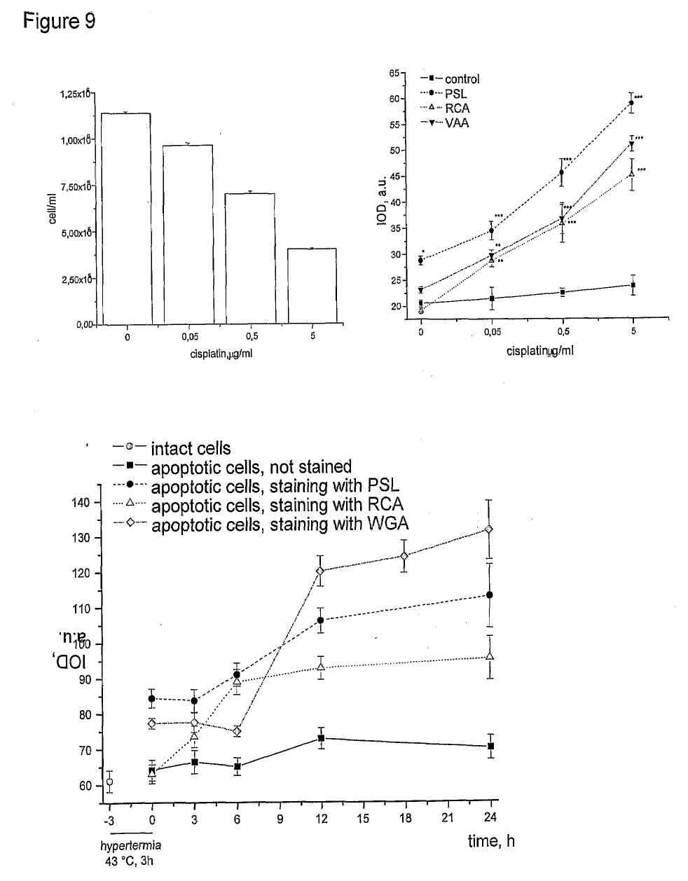

Dose-dependent effect of apoptosis-inducing agent (cisplatin) on glycoprotein expression during apoptosis was demonstrated in murine leukemia L1210 cells (FIG. 9A). The inventors previously showed that cisplatin used in the same concentrations induced apoptosis (DNA fragmentation) in these cells (Stoika R, Yakymovych M, Yakymovych I, Chekhun V. Cisplatin resistant derivatives of murine L1210 leukemia cells are not susceptible to growth-inhibiting and apoptosis-inducing actions of transforming growth factor b1. Anti-Cancer Drugs. 1999; 10: 457-463). An increase in cisplatin concentration led to a decrease in live cell number (FIG. 9A), and to an increase in expression of mannose- and galactose-rich glycoprotein (FIG. 9B), measured by lectinocytochemical analysis using HRP-labeled lectins (PSL and RCA or VAA, respectively).

### Example 23

### Time-Dependent Effect of Apoptosis Inducing Action on Glycoprotein Expression

Time-dependent effect of apoptosis inducing action on glycoprotein expression was studied using L929 cells subjected to hyperthermia (43° C., 3 h) (FIG. 9C). The earliest marked increase in glycoprotein expression was observed in 6 h after starting apoptosis induction for glycoproteins, that bound RCA, and in 12 h—for glycoproteins binding PSL and WGA.

### Example 24

### Effect of Internalization on Expression of Plasma Membrane Glycoproteins Characteristics for Apoptotic Cells

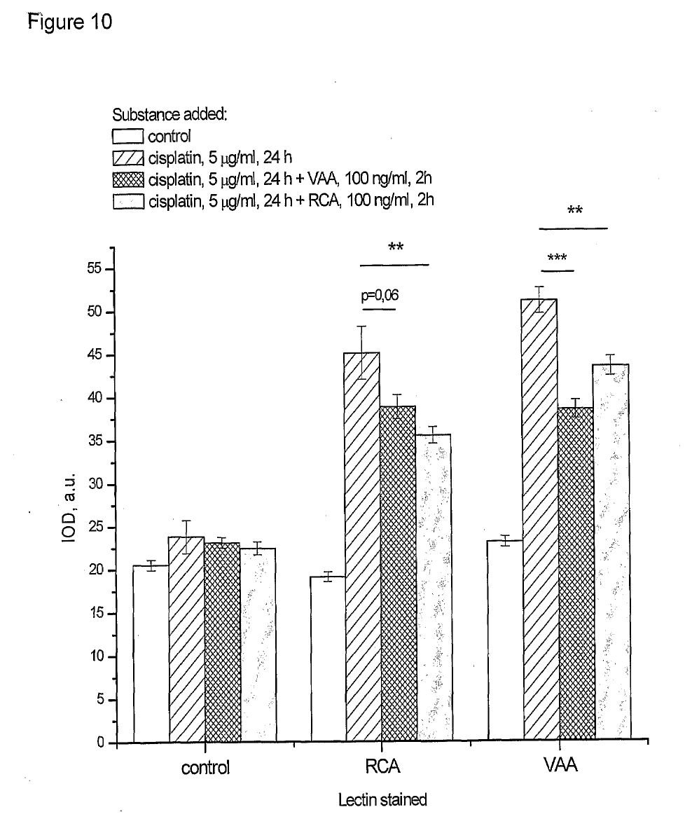

Pretreatment of L1210 cells for 2 h with RCA, VAA, WGA, PSL and.PMRL lectins and subsequent labeling of these cells with an appropriate HRP-labeled lectin demonstrated a decrease (p<0.05 in all cases) in lectin binding with pretreated cells in comparison to untreated ones. That could be explained by internalization of glycoprotein receptors for the corresponding lectins. It should be noted that pretreatment of cells with RCA lectin decreased binding for not only HRP-labeled RCA lectin, but also for HRP-labeled VAA lectin, that is similar in its carbohydrate specificity to RCA, and vice versa, cell pretratment with VAA decreased their binding of VAA and RCA (FIG. 10).

### Example 25

### Lectin-Stimulated Agglutination of Normal and Apoptotic Cells

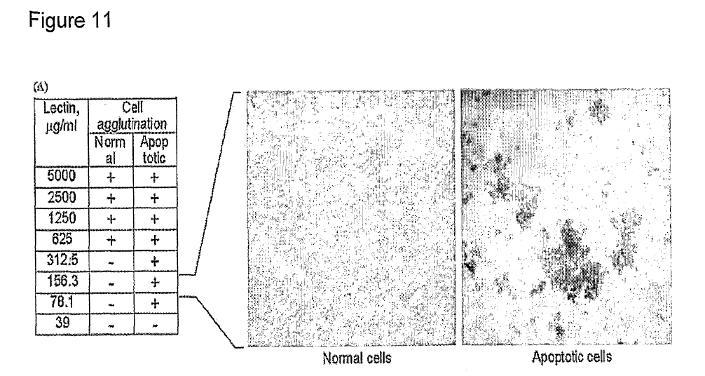

Agglutination of normal and apoptotic (FIG. 11) murine leukemia L1210 cells demonstrated differences in minimal lectin concentration needed to induce noticeable agglutination. Mannose-specific PMRL and PSL lectins caused agglutination of normal cells in concentrations of 625 and 1000 μg/ml, respectively and agglutination of apoptotic cells in concentrations of 78 and 125 μg/ml, respectively (FIG. 11). Thus, lectin concentration needed for agglutination of apoptotic cells was approximately equal to and/or equal to ⅛ (625 against 78 and 1000 against 125) of that needed to agglutinate the non-apoptotic cells. It is believed that there is an 8-fold increase in mannose-containing glycoconjugate expression in the apoptotic cells comparing to intact cells. Similar results were obtained for galactose-specific lectins.

### Example 26

### Lectin-Affinity Isolation of Apoptotic Cells

Isolation of apoptotic L1210 cells was carried out using PSL-conjugated coarse-grained agarose (FIG. 12A). Separation of intact cells was carried out by washing the affinity column with double volume of TSB, pH 7.4; separation of apoptotic cells was carried out by washing the PSL-affinity column with double volume of 0.05 M borate buffer, pH 8.0. Separation of intact cell population resulted in obtaining a fraction of PSL-agarose-bound cells (positive) that constituted 1.45±0.44% of total cell population, and negative fraction (98.5+0.45% of total cell population). When apoptosis was induced by cisplatin (5 μg/ml, 24 h) the yield of apoptotic cells was 7.55±0.55% of total cell population (positive fraction), while the negative fraction yield was 92.35±0.55%. Subsequent epifluorescent microscopy (FIG. 12B) of isolated cell suspensions using acridine orange staining and light microscopy with hematoxylin staining (not shown) allowed classifying cells of negative fraction as “intact” and cells of positive fraction as “apoptotic” ones. An increased expression of mannose-rich glycoconjugates may be used for isolating a cell population enriched with apoptotic cells.

### Example 27

### Lectin-Induced Agglutination of Intact and Apoptotic L1210 Cells.

The inventors also tested additional lectins; namely, VAA, SNA, PMRL, LVA, PLA, L-fucose specific lectin from river perch, HPL, RCA, and PSL for their ability to agglutinate intact and apoptotic cells.

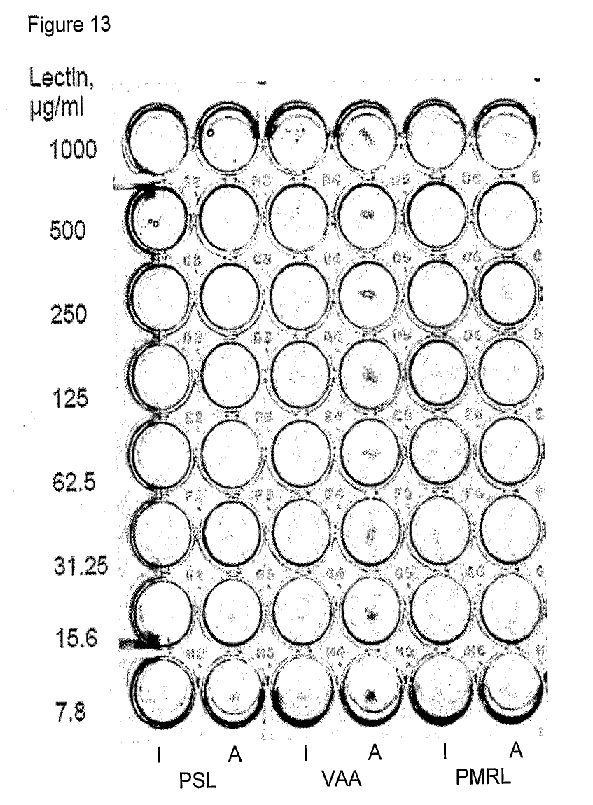

Similar testing in 96-well immunological plates was carried out. It was found that lectin VAA agglutinated the apoptotic murine leukemia L1210 cells in the concentration of 7.8 μg/ml, and intact cells in a concentration of 1,000 μg/ml, providing a 128-fold (1,000/7.8) concentration difference in agglutination (FIG. 13). PMRL lectin provided a 4-fold concentration difference in agglutination of intact and apoptotic L1210 cells. Other tested lectins did not provide noticeable agglutination of intact or apoptotic cells. It should be noted that the highest lectin concentration tested (1,000 μg/ml) is quite low for the agglutination reaction; while almost all tested lectins in concentrations of 10,000 pg/ml agglutinated both intact and normal cells. The inventors believe that lower concentration of lectin tested for agglutination (in a range of 1,000 to 7.8 μg/ml with a step/2) was providing a better specificity.

### Example 28

### Lectin-Induced Agglutination of Intact and Apoptotic Jurkat Cells

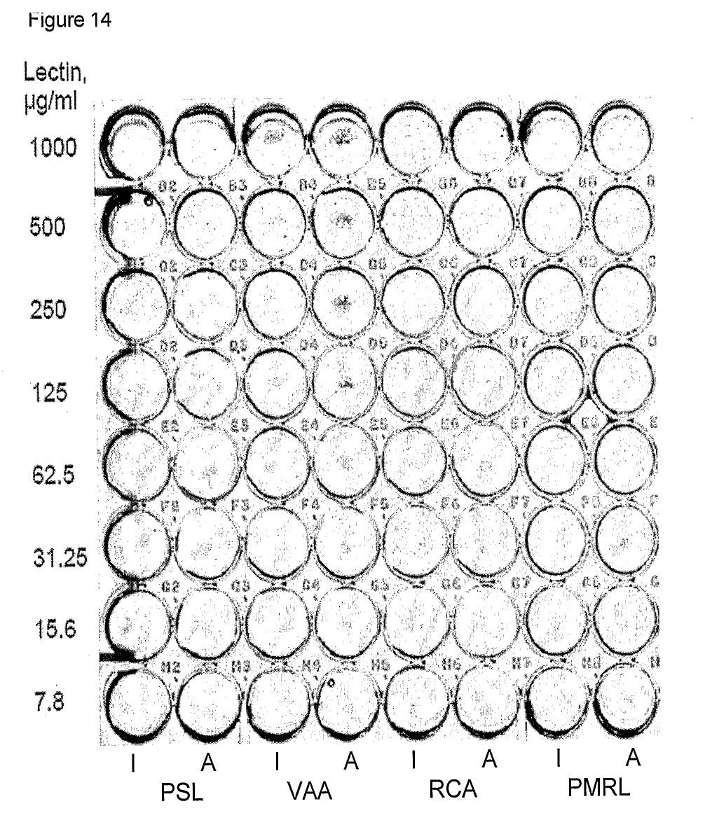

A human Jurkat T cell line was used. The ability of PSL, VAA, RCA and PMRL lectins to induce agglutination was also tested (FIG. 14). It was found that VAA lectin effectively discriminated between intact and apoptotic cells, agglutinating intact cells in concentration of 1,000 μg/ml, while the apoptotic cells were agglutinated by its concentration 62.5 μg/ml, thus providing a 16-fold concentration difference in the agglutination reaction.

### Example 29

### Lectin-Induced Agglutination of Freshly Isolated Human Peripheral Blood Lymphocytes

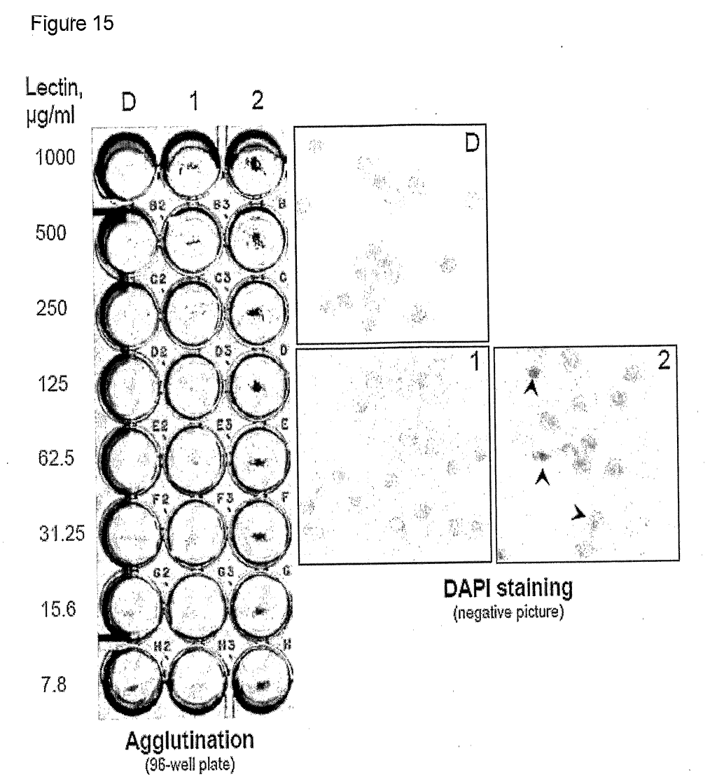

The approach the inventors developed for apoptotic cell detection in vitro was further adapted for apoptosis detection in freshly isolated peripheral blood lymphocytes obtained from “healthy” donors and patients with specific autoimmune diseases. In this case VAA lectin was used. It was shown that lectin concentration of 1,000 μg/ml did not induce noticeable agglutination of lymphocytes of healthy donors (FIG. 15). DAPI staining of those lymphocytes revealed that less than 1% of cells possessed condensed or fragmented nuclei, characteristic for the apoptotic cells. At the same time, the lymphocytes of patient N. G. 1 diagnosed for “rheumatoid arthritis” were agglutinated by 1000 μg/ml of VAA lectin. DAPI staining revealed that 1.1% of lymphocytes were apoptotic here. Lymphocytes of patient T. O. 2 with diagnosis “polyosteoarthritis” were agglutinated by 15.6 μg/ml of VAA lectin and DAPI staining revealed that 6.7% of the lymphocytes were apoptotic (FIG. 15). Thus, VAA concentration needed to agglutinate lymphocytes of patient 2 was 64 times less than that needed for agglutination of lymphocytes of “healthy” donor. A strong negative correlation (r=−0,882) was observed between the number of apoptotic cells and minimal lectin concentration, needed for cell agglutination.

Lymphocytes isolated from the peripheral blood of 50 autoimmune disease patients were tested. In 93.75% cases showing apoptosis (15 of 16 patients), a strong positive correlation was found between cell agglutination by VAA lectin and the number of apoptotic cells, revealed by DAPI staining.

### Example 30

### Lectin-Induced Agglutination of Freshly Isolated Human Peripheral Blood Lymphocytes Before and After Chemotherapeutic Treatment

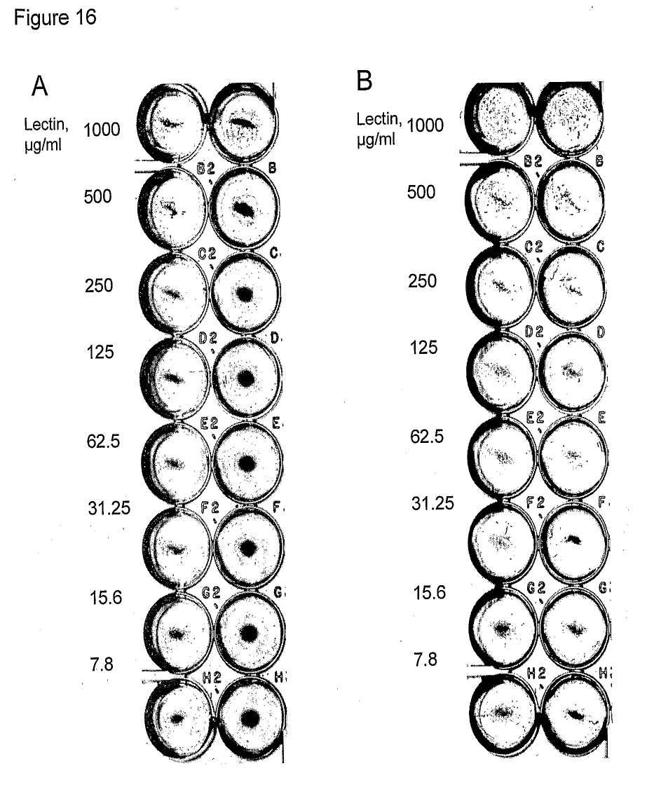

The approach the inventors developed for apoptotic cell detection in vitro was further adapted for apoptosis detection in fresh isolated peripheral blood lymphocytes obtained from “healthy” donors and patients with specific autoimmune diseases before and after the chemotherapy. It was shown that lectin concentration of 1000 μg/ml did not induce noticeable agglutination of lymphocytes of healthy donor (D) (FIG. 16, A and B). DAPI staining of those lymphocytes revealed that less than 1% of cells possessed condensed or fragmented nuclei, characteristic for the apoptotic cells. Lymphocytes of patient “V.P. 1,” diagnosed for “active articular form of polyarthritis” were agglutinated by 7.8 μg/ml of VAA lectin. DAPI staining revealed that 5.74% of lymphocytes were apoptotic (FIG. 16A). After a 14-day course of chemotherapy, the lymphocytes of patient V.P. 1 were agglutinated by 62.5 μg/ml of VAA lectin. DAPI staining revealed that the 3.85% of lymphocytes were apoptotic (FIG. 16B). Thus, anti-arthritis chemotherapy during the 14 days led to the increase in minimal VAA concentration needed for agglutination of isolated lymphocytes and to simultaneous decrease in number of apoptotic cells in patient's blood as well as to improvement of other clinical parameters.

### Example 31

At least two lectins with the same or similar carbohydrate specificity; for example PSL and GNA, are used for studying mannose-containing glycoconjugate expression, and RCA and VAA are used for detection of galactose-containing glycoconjugate expression.

### Example 32

### Quantification of Live and Apoptotic Cells by Agglutination Method

Apoptosis of L1210 cells was induced by cisplatin used in different concentrations, namely 0.05, 0.5 and 5 μg/ml, for 24 hours. The percentage of live cells in each population after apoptosis induction was calculated by the trypan blue exclusion test. A number of the apoptotic cells equals:

% apoptotic cells=100% cells−% live cells

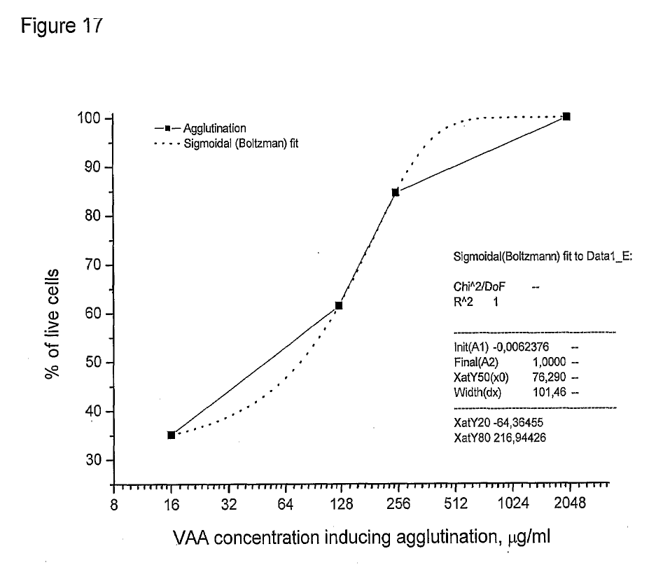

The concentration of VAA lectin needed to agglutinate cells in their population was detected. The dependence of a percentage of live cells in population upon specific lectin concentration needed for agglutination is shown (see FIG. 17). A sigmoidal fit of the dependence was proposed by the inventors for a description of that dependence. The described dependence includes a plateau, meaning that in order to detect a population with 90% live cells (10% apoptotic), it is usually necessary to use VAA in 250 or 500 μg/ml concentration, while VAA in concentration 2,000 μg/ml will be high enough to agglutinate the intact cells (100% alive, 0% apoptotic). It should be noted, that the presented dependence may vary depending on target cell type, apoptosis inducer, duration of apoptosis induction, or time after its induction, and specific lectin used. Thus, the presented dependence can serve as an example. From this example, each specific case of agglutination testing a particular calibration may be performed by one skilled in the art.

### Example 33

### Detection of Apoptotic Cells by Means of Fluorescent Microscopy and Fluorescent Conjugates of Lectins

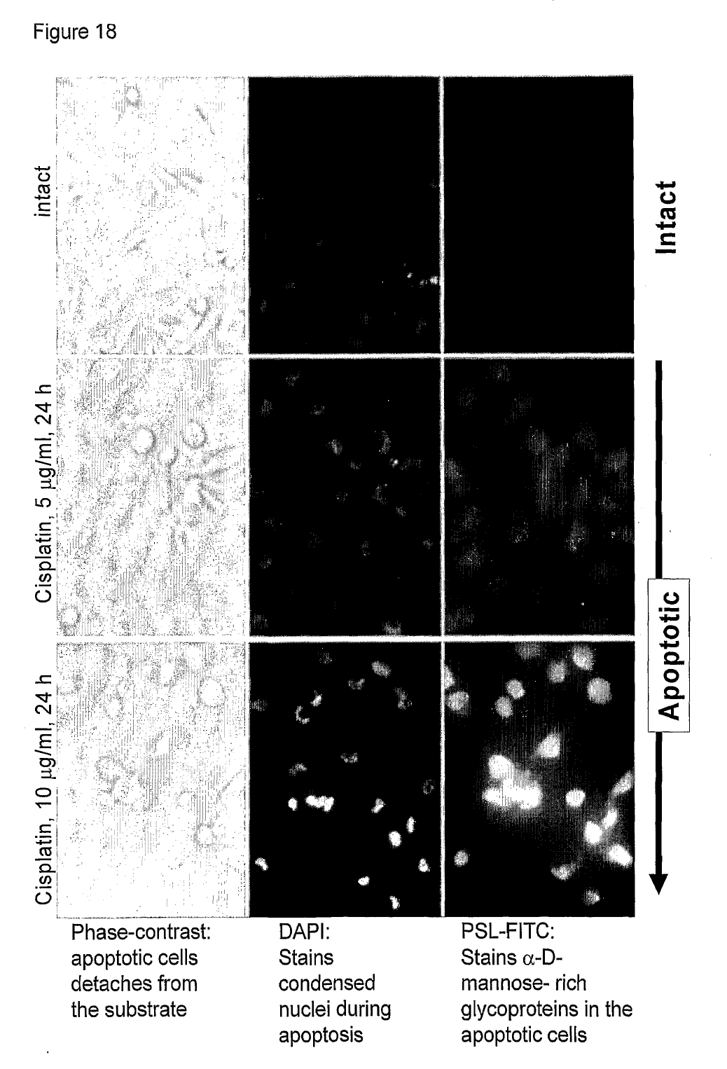

FITC-PSL conjugate was used for the detection of apoptotic cells of human lung carcinoma A549 cells. Cisplatin, which is a potent and dose-dependent inducer of apoptosis, was used to cause the programmed cell death. Untreated cells (see FIG. 18, upper row) did not bind labeled PSL lectin, and at the same time no signs of apoptosis were found in cell populations: cell nuclei were not fragmented and/or condensed while stained with DAPI and cells were firmly attached to the substrate (revealed by phase-contrast microscopy). Treatment of A549 cells with 5 μg/ml cisplatin for 24 hours lead to the loss of firm contact between some cells and substrate with simultaneous nuclei condensation of the cells, indicating the beginning of apoptotic cell death. Cells in this population also bound FITC-PSL significantly stronger when compared to untreated cells (see FIG. 18 middle row). Treatment of human lung carcinoma A549 with 5 μg/ml cisplatin for 24 hours caused apoptosis in almost all cells (cells lost the contact with the substrate and almost all nuclei were condensed and/or fragmented when stained with DAPI). Binding of FITC-PSL was significantly higher in these cells when compared to untreated cells or cells with the onset of apoptosis (see FIG. 18 lower row). In all cases the presence of apoptosis was also confirmed by DNA fragmentation, revealed by DNA gel electrophoresis (data not shown). This example illustrates the efficacy and specificity of fluorescent lectin staining in detection of apoptotic cells.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for detecting apoptotic cells in a sample of cells, comprising:
providing a lectin that possesses at least two carbohydrate-recognition domains; and
adding a quantity of the lectin to the sample of cells,
wherein the observation of agglutinating cells in the sample indicates the presence of apoptotic cells.

2. The method of claim 1, wherein the quantity of the lectin is less than a quantity of lectin that is capable of causing agglutination of intact cells.

3. The method of claim 1, wherein the lectin is labeled with a label selected from the group consisting of enzymatic label, biotin, fluorescent and combinations thereof, and the method further comprise detecting the presence of the label, wherein the presence of the label indicates the presence of apoptotic cells.

4. The method of claim 1, further comprising:
determining a minimum quantity of lectin that causes agglutination of the cells; and
comparing the minimum quantity of lectin to predetermined quantities of lectin that cause agglutination of intact cells and apoptotic cells, wherein if the minimum quantity of lectin is less than the predetermined quantity of lectin that causes agglutination of intact cells, the presence of apoptotic cells is indicated.

5. The method of claim 1, wherein the lectin is capable of simultaneously binding at least two cells.

6. The method of claim 1, wherein the lectin is capable of binding to an α-D-mannose-rich glycoprotein, a β-D-galactoste-rich glycoprotein, or both.

7. The method of claim 1, wherein the lectin is selected from the group consisting of lectins from Pisum sativum (PSL), Polygonatum multiforum (PMRL), Galanthus nivalis (GNA), Ricinus communis (RCA-120), Viscum album (VAA), and combinations thereof.

8. The method of claim 1, wherein the lectin is from Viscum album.

9. The method of claim 1, wherein detecting apoptotic cells comprises detecting apoptotic cells after about 12 hours after induction of apoptosis.

10. The method of claim 4, wherein the lectin is from Pisum sativum (PSL) and the predetermined quantity for intact cells is about eight times higher than the predetermined quantity for apoptotic cells.

11. The method of claim 4, wherein the lectin is from Polygonatum multiforum (PMRL) and the predetermined quantity for intact cells is from about four to about eight times higher than the predetermine quantity for apoptotic cells.

12. The method of claim 4, wherein the lectin is from Viscum album (VAA) and the predetermined quantity for intact cells is from about 4 times to about 128 times higher than the predetermined quantity for apoptotic cells.

13. The method of claim 1, wherein the sample of cells comprises human lymphocytes.

14. A method of quantifying the amount of apoptotic cells in a sample of cells, comprising:
providing a lectin that possesses at least two carbohydrate-recognition domains;
determining a minimum quantity of the lectin that is capable of causing the sample of cells to agglutinate; and
comparing the minimum quantity of lectin to predetermined quantities of lectin that cause agglutination of intact cells and apoptotic cells in various stages after induction of apoptosis to determine the quantity of apoptotic cells in the sample of cells.

15. The method of claim 14, wherein the lectin is labeled with a label selected from the group consisting of enzymatic label, biotin, fluorescent and combinations thereof, and the method further comprise detecting the presence of the label, wherein the presence of the label indicates the presence of apoptotic cells.

16. The method of claim 14, wherein the lectin is capable of simultaneously binding at least two cells.

17. The method of claim 14, wherein the lectin is capable of binding to an α-D-mannose-rich glycoprotein, α-D-galactoste-rich glycoprotein, or both.

18. The method of claim 14, wherein the lectin is selected from the group consisting of lectins from Pisum sativum (PSL), Polygonatum multiforum (PMRL), Galanthus nivalis (GNA), Ricinus communis (RCA-120), Viscum album (VAA), and combinations thereof.

19. The method of claim 14, wherein the lectin is from Viscum album (VAA).

20. The method of claim 14, wherein quantifying the amount of apoptotic cells comprises quantifying the amount of apoptotic cells after about 12 hours after induction of apoptosis.

21. The method of claim 14, wherein predetermined quantities of lectin that cause agglutination of intact cells and apoptotic cells in various stages after induction of apoptosis are determined by correlating quantities of lectin that cause agglutination of control samples of cells within known amounts of apoptotic cells.

22. A method for isolating apoptotic cells from a sample of cells, comprising:
providing a conjugated lectin;
contacting the sample of cells to the conjugated lectin to generate a fraction of cells that are bound to the conjugated lectin and a fraction of cells that are not bound to the conjugated lectin; and
separating the fraction of cells that are bound to the conjugated lectin from the conjugate to produce a fraction of cells comprising the apoptotic cells.

23. The method of claim 22, wherein the conjugated lectin is a lectin-conjugated support medium.

24. The method of claim 22, wherein the conjugate is a label selected from the group consisting of enzymatic label, biotin, fluorescent and combinations thereof, and the method further comprise detecting the presence of the label, wherein the presence of the label indicates the presence of apoptotic cells.

25. The method of claim 22, wherein the lectin is capable of simultaneously binding at least two different cells.

26. The method of claim 22, wherein the lectin is capable of binding to an α-D-mannose-rich glycoprotein, a β-D-galactose-rich glycoprotein, or both.

27. The method of claim 22, wherein the lectin is selected from the group consisting of lectins from Pisum sativum (PSL), Polygonatum multiforum (PMRL), Galanthus nivalis (GNA), Ricinus communis (RCA-120), Viscum album (VAA), and combinations thereof.

28. A kit for the detection and/or quantification of apoptotic cells in a sample of cells, comprising:
a quantity of a lectin that possesses at least two carbohydrate-recognition domains; and
instructions to use the quantity of lectin to detect and/or quantify apoptotic cells.

29. The kit of claim 28, wherein the lectin is capable of simultaneously binding at least two cells.

30. The kit of claim 28, wherein the lectin is capable of binding to an α-D-mannose-rich glycoprotein, a β-D-galactose-rich glycoprotein, or both.

31. The kit of claim 28, wherein the lectin is selected from the group consisting of lectins from Pisum sativum (PSL), Polygonatum multiforum (PMRL), Galanthus nivalis (GNA), Ricinus communis (RCA-120), Viscum album (VAA), and combinations thereof.

32. The kit of claim 28, wherein the lectin is from Pisum sativum (PSL) or Viscum album (VM).

33. The kit of claim 28, wherein the instructions to use the quantity of lectin to detect apoptotic cells comprise instructions to:
add a quantity of the lectin to the sample of cells; and
detect the presence of agglutination of cells in the sample,
wherein the quantity of lectin is less than a quantity of lectin that is capable of causing agglutination of intact cells and the presence of agglutination of cells indicates the presence of apoptotic cells.

34. The kit of claim 33, wherein the instructions further comprise instructions to:
determine a minimum quantity of lectin that causes agglutination of the cells; and
compare the minimum quantity of lectin to predetermined quantities of lectin that cause agglutination of intact cells and apoptotic cells,
wherein the minimum quantity of lectin that is less than the predetermined quantity of lectin that causes agglutination of intact cells indicates the presence of apoptotic cells.

35. The kit of claim 34, wherein the lectin is from Pisum sativum (PSL) and the predetermined quantity for intact cells is about eight times higher than the predetermined quantity for apoptotic cells.

36. The kit of claim 34, wherein the lectin is from Polygonatum multiforum (PMRL) and the predetermined quantity for intact cells is from about four to about eight times higher than the predetermine quantity for apoptotic cells.

37. The kit of claim 34, wherein the lectin is from Viscum album (VAA) and the predetermined quantity for intact cells is from about 4 times about 128 times higher than the predetermined quantity for apoptotic cells.

38. The kit of claim 28, wherein the instructions to use the quantity of lectin to quantify apoptotic cells comprise instructions to:
determine a minimum quantity of the lectin that is capable of causing the sample of cells to agglutinate; and
compare the minimum quantity of lectin to a predetermined quantities of lectin that cause agglutination of intact cells and apoptotic cells in various stages after induction of apoptosis to determine the quantity of apoptotic cells.

39. The kit of claim 38, wherein the predetermined quantities of lectin that cause agglutination of intact cells and apoptotic cells in various stages after induction of apoptosis are determined by correlating quantities of lectin that cause agglutination of control samples of cells within known amounts of apoptotic cells.

40. A kit for isolating apoptotic cells from a sample of cells, comprising:
a quantity of conjugated lectins; and
instructions to use the quantity of conjugated lectins to isolate apoptotic cells.

41. The kit of claim 40, wherein the conjugated lectin is a lectin-conjugated support medium.

42. The kit of claim 40, wherein the instructions comprise instructions to:
contact the sample of cells to the conjugated lectin to generate a fraction of cells that are bound to the conjugated lectin and a fraction of cells that are not bound to the conjugated lectin;
separate the fraction of cells that are bound to the conjugated lectin and the fraction of cells that are not bound to the conjugated lectin; and
separate the fraction of cells that are bound to the conjugated lectin from the conjugated lectin.

43. The kit of claim 42, wherein the conjugate is a label selected from the group consisting of enzymatic label, biotin, fluorescent and combinations thereof, and the instructions further comprise instructions to detect the presence of the label, wherein the presence of the label indicates the presence of apoptotic cells.

44. The kit of claim 40, wherein the lectin is capable of simultaneously binding at least two cells.

45. The kit of claim 40, wherein the lectin is capable of binding to an α-D-mannose-rich glycoprotein, a β-D-galactose-rich glycoprotein, or both.

46. The kit of claim 40, wherein the lectin is selected from the group consisting of lectins from Pisum sativum (PSL), Polygonatum multiforum (PMRL), Galanthus nivalis (GNA), Ricinus communes (RCA-120), Viscum album (VAA), and combinations thereof.
